(19) United States
(12) Patent Application Publication
(10) Pub. No.: US 2026/0055468 A1
(43) Pub. Date: Feb. 26, 2026

(54) TREATMENT AND METHOD FOR PREDICTING DISEASE INCIDENCE, PROGRESSION, AND PROGNOSIS OF GLIOMA

(71) Applicant: Ramot at Tel-Aviv University Ltd., Tel Aviv (IL)

(72) Inventors: Noam SHOMRON, Tel Aviv (IL); Ori MAYER, Tel Aviv (IL)

(21) Appl. No.: 19/102,806

(22) PCT Filed: Sep. 22, 2023
(86) PCT No.: PCT/IB2023/059395

§ 371 Date: Feb. 10, 2025

Related U.S. Application Data
(60) Provisional application No. 63/409,058, filed on Sep. 22, 2022.

(51) Int. Cl. C12Q 1/6886; G01N 33/574; G01N 33/68

(52) U.S. Cl. CPC C12Q 1/6886; G01N 33/57496; G01N 33/6887; C12Q 2600/158; G01N 2333/4712

(57) ABSTRACT

A method for predicting disease incidence, progression, aggressiveness, and prognosis of brain tumors including gliomas and astrocytomas using palladin as a marker. Palladin expression is also disclosed as a therapeutic target for the treatment of brain tumors, in addition to a method of treating brain tumors by regulating and/or modifying and/or deleting palladin expression. In embodiments, palladin is targeted by therapeutic agents that are systemically or locally administered. Targeted glioma tumors include astrocytomas, oligodendrogliomas, and glioblastomas. The disclosure provides that palladin expression can be linked to adult astrocytoma progression and is associated with a worsening prognosis.

### FIELD

This disclosure relates to methods of detecting or diagnosing brain tumors in a subject in need via measurement of PALLD mRNA levels and therapeutic agents to treat the same.

### BACKGROUND

Palladin, encoded in the PALLD gene, is a structural protein widely expressed in mammalian tissues, and plays a pivotal role in cytoskeletal dynamics and motility in healthy and diseased tissues. In the central nervous system (CNS) where palladin is expressed in the neural plate, neural progenitor cells, cortical neurons, and astrocytes, palladin is involved in embryonic development, neuronal maturation, the cell cycle, differentiation, and apoptosis. It is localized to both highly motile and actin rich structures such as stress fibers, focal adhesions, dorsal ruffles, podosomes, Z-discs, invadopodia and filopodia. Palladin acts as a major scaffolding protein by recruiting other actin-related proteins, such as profilin, VASP, α-actinin, ezrin, PDLIM1, Eps8 and LASP-1. In addition, it promotes actin filament nucleation and crosslinking, supporting stronger fibers in higher numbers. Taken together, this indicates that palladin is a major player in cytoskeletal dynamics. However, the role of palladin in brain tumors has previously been unknown.

Brain tumors comprise over 100 types of masses, differing in location, patient age group, histological and immunohistochemical characteristics, prognosis, and treatment. Gliomas, which originate from the brain's support cells, or neuroglia, comprise 23-25% of all brain tumors and 80% of malignant brain tumors. The World Health Organization (WHO) classifies glioma tumors according to their molecular, histological, and immunohistochemical characteristics as well as whether the tumor is diffuse or circumscribed, and whether it occurs in an adult or pediatric patient. To diagnose central nervous system (CNS) tumors a pathologist determines the histology of the tumor; grades it 1-4 and reports any molecular characteristics of interest. Finally, by taking into account all the features of the lesion an integrated diagnosis is assigned following the WHO CNS5 guidelines. Typical survival time ranges from 1-10 years with glioblastomas, the most common form of glioma, having a 5-year survival rate of only 6.8%.

The current leading prognostic factors of glioma are age, Karnofsky performance score (KPS), and tumor grade. The number of glioma lesions and the degree of surgical resection also impact prognosis. Several genetic alterations serve as prognostic factors for glioma. Loss of heterozygosity of 1p/19q is considered a favorable prognostic factor, though this association is stronger in oligodendrogliomas than in astrocytomas and glioblastomas, and is therefore also used in diagnosis. Gain of function mutations in the TP53 gene causing overexpression of p53 protein is an adverse prognostic factor associated with shorter overall survival. Isocitrate dehydrogenase 1 (IDH1) and 2 (IDH2) mutations are favorable prognostic factors and are used to diagnose astrocytoma and oligodendrogliomas, whereas IDH-wildtype is associated with lower overall survival and characterizes glioblastomas. Promoter methylation of 06-methylguanine DNA 58 methyltransferase (MGMT) is another favorable prognostic factor that is associated with more sensitivity to alkylating agents in chemotherapy. Lastly, ATRX mutations, which occur most often in astrocytoma, are associated with wildtype 1p/19q and mutations in IDH1/2 and TP53, and may be involved in alternative lengthening of telomeres, contributing to genomic instability.

The current standard of care for gliomas is surgical resection, followed by chemotherapy with temozolomide (TMZ) and radiation therapy. Tumor treating fields (TTFields) are alternating electric fields that stunt tumor growth by interfering with the cell cycle. Clinical trials have shown that TTFields in combination with TMZ improve the overall median survival of patients with glioblastoma by 5 months compared to TMZ alone. The drug bevacizumab employs a humanized antibody that targets human vascular endothelial growth factor, resulting in decreased tumor vascularization and consequently, reduced tumor proliferation. Despite advances in treatments, high-grade gliomas (HGG) remain largely incurable. Thus, advanced methods for treating and predicting disease incidence, progression, and prognosis of gliomas are needed.

The present disclosure includes an analysis of clinical data and bulk and single-cell gene expression to provide insight into palladin's role in glioma. According to the disclosure, palladin expression is linked to adult glioma progression and a worsening prognosis. Results were validated using IHC staining of tumor samples together with qRT-PCR of glioma cell lines. It was determined that wild-type palladin-4 is overexpressed in adult gliomas and is correlated with a decrease in survival.

Astrocytoma, a glioma originating from astrocytes, is the most prevalent malignant adult brain tumor. Palladin expression is most prominent in astrocytoma. The findings of the present disclosure indicate that palladin expression can be linked to adult glioma and astrocytoma progression and is useful for determining, inter alia, disease aggressiveness and prognosis. According to the disclosure, palladin expression outperforms current clinically used prognostic markers and can be used in a method for predicting disease incidence, progression, aggressiveness, and prognosis of brain tumors including glioblastomas and astrocytoma. According to the present disclosure, palladin expression is also a therapeutic target for the treatment of brain tumors.

### SUMMARY

Glioma is a tumor originating from cells supporting the brain and represents a major health challenge. Astrocytoma, a glioma originating from astrocytes, is the most prevalent malignant adult brain tumor. Palladin is a structural protein widely expressed in mammalian tissues and has a pivotal role in cytoskeletal dynamics in health and disease. The present disclosure provides insight into palladin's role in brain tumors such as gliomas and astrocytoma, and solves the issue of drugging these difficult target.

According to the disclosure, palladin expression can be linked to adult astrocytoma progression and is associated with a worsening prognosis. Overall, the disclosure introduces a method of using palladin as a marker for predicting disease incidence, progression, aggressiveness, and prognosis of brain tumors such as gliomas and astrocytoma, as well as a future therapeutic target. In other words, the invention solves the problem of a lack of disease markers for gliomas. This invention also addresses the problem of a lack of effective therapeutic agents to treat gliomas and astrocytoma, among other diseases. Importantly, the disclosure provides that overexpression of wild-type palladin's isoform 4, originating mostly from the malignant cell population, is involved in the progression of aggressive adult glioma tumors, and that this expression correlates to decreased survival. This results permit the use of palladin as a diagnostic and prognostic marker, as well as a therapeutic target.

In some embodiments, a method of treating brain tumors in a subject is disclosed. In embodiments, detecting or diagnosing glioma in the subject includes: a) measuring PALLD mRNA levels in a plasma sample of the subject; correlating the PALLD mRNA levels to PALLD mRNA plasma levels corresponding to a likelihood of having a specific type of glioma; making a diagnosis based on the PALLD mRNA levels of the plasma sample as compared to the expected PALLD mRNA plasma levels corresponding to a likelihood of having a specific type of glioma; and b) treating subject diagnosed with specific type of glioma or having the likelihood of developing specific type of glioma with at least one therapeutic agent for treating glioma. In other embodiments, disclosed is a method of treating brain tumors wherein the at least one therapeutic agent (e.g., miR-96 and miR-182, or the like) causes downregulation of palladin within targeted tissue of a subject. In yet another embodiment, the at least one therapeutic agent causing downregulation of palladin comprises miR-96 and miR-182, wherein the miR-96 and miR-182 are bound to gold nanoparticles and/or and are further embedded in a hydrogel.

In another embodiment, the therapeutic agent includes at least one miRNA molecule selected from the group consisting of miR-96 and miR-182, or at least one vector expressing or encoding the same, for use in reducing or preventing the specific type of glioma. In other embodiments, the therapeutic agent is formulated for systemic administration, local administration, intra-tumor administration, enteral administration, oral administration, sublingual and buccal administration, rectal administration, intravenous administration, intramuscular administration, and/or subcutaneous administration. In some embodiments, the glioma comprises a cancer associated with abnormal palladin expression and/or activity. In other embodiments, the glioma is an ependymoma, the glioma is selected from the group consisting of astrocytoma or oligodendroglioma, or the glioma comprises a glioblastoma (or another high-grade glioma originating in astrocytes). In other embodiments, the subject is at risk of developing metastasis and the administering is carried out prior to metastasis formation. In yet another embodiment, the subject has already developed metastases and the administering is carried out after metastasis formation.

In other embodiments, a method of treating brain tumors is disclosed wherein a therapeutic agent inhibits the binding of palladin to actin cytoskeleton (e.g., actin binding proteins, Jasplakinolide, Latrunculin B, and the like). In embodiments, over 150 actin-binding proteins (ABPs) are known that may influence localization, polymerization dynamics, crosslinking, and organization of actin. These ABPs have various means of regulating actin, which include the ability to sequester monomeric actin (G-actin), nucleate filament formation, sever filamentous actin (F-actin), generate branched arrays of actin, and cap actin filaments. In embodiments, glioblastoma tumors express approximately 150% and 70% more palladin than oligodendroglioma, and astrocytoma tumors, respectively (P<0.0001 and P<0.0001).

In still other embodiments, a method of detecting or diagnosing glioma tumor type in a subject is disclosed. Said method includes the steps of measuring PALLD mRNA levels in a plasma sample of the subject; correlating the PALLD mRNA levels to PALLD mRNA plasma levels corresponding to a likelihood of having a specific type of glioma; and making a diagnosis based on the PALLD mRNA levels of the plasma sample as compared to the expected PALLD mRNA plasma levels corresponding to a likelihood of having a specific type of glioma. In other embodiments, said method of detecting or diagnosing glioma comprises an adult-type diffuse glioma. In another embodiment, a method is disclosed delineating brain tumor tissue from surrounding brain, the method comprising injecting a palladin-associating fluorophore or prodrug comprising a palladin-associating fluorophore into a subject for fluorescence-guided neurosurgery.

### BRIEF DESCRIPTION OF THE DRAWINGS

FIG. 1A shows palladin expression in healthy versus tumor samples, including tumor samples from adrenal, bile duct, bladder, brain, and breast cancers. FIG. 1B shows palladin expression in healthy versus tumor samples, including tumor samples from cervix, colon, endometrium, esophagus, and head/neck cancers. FIG. 1C shows palladin expression in healthy versus tumor samples, including tumor samples from kidney, liver, lung, ovary, and pancreas cancers. FIG. 1D shows palladin expression in healthy versus tumor samples, including tumor samples from paraganglia, prostate, rectum, skin, and soft tissue/bone cancers. FIG. 1E shows palladin expression in healthy versus tumor samples, including tumor samples from stomach, testis, thymus, thyroid, and uterus cancers. In summary, FIGS. 1A-1E show that wild-type palladin-4 mRNA is overexpressed in adult gliomas and is correlated with decreased survival.

FIG. 2A shows overall survival of the cholangiocarcinoma cohort. FIG. 2B shows overall survival of the glioma cohorts. FIG. 2C shows overall survival of the breast cancer cohorts. FIG. 2D shows overall survival of the hepatocellular carcinoma cohorts. FIG. 2E shows overall survival of the pancreatic adenocarcinoma cohorts. FIG. 2F shows overall survival of the glioma cohorts. FIG. 2G shows overall survival of the stomach adenocarcinoma cohorts. FIG. 2H shows overall survival of the thyroid carcinoma cohorts. In summary, FIGS. 2A-2H shows overall survival of all palladin overexpressing tumors in FIGS. 1A-1E, stratified into three groups based on expression level.

FIG. 3 shows a schematic representation of palladin's complete coding transcripts, their protein products, and somatic mutations.

FIG. 4A shows expression levels the coding palladin isoform ENST00000505667.6. FIG. 4B shows expression levels the coding palladin isoform ENST00000261509.1 FIG. 4C shows expression levels the coding palladin isoform ENST00000512127.5. FIG. 4D shows expression levels the coding palladin isoform ENST00000507735.5.

FIG. 5 provides a comparison of palladin expression in fetal brains and adult brains.

FIG. 6 is a qRT-PCR analysis of palladin expression in murine glioblastoma cells and normal brain tissue.

FIG. 7A is an analysis of tissue microarray stained with an a-palladin antibody (sample type vs. proportion stained). Staining is shown in respect to sample type (left column), histological grade (middle column), and histological subtype (right column). Proportion (top row) and intensity (bottom row) are shown of stained nuclei (black), membrane (red), and cytoplasm (green). FIG. 7B is an analysis of tissue microarray stained with an a-palladin antibody (WHO grade vs. proportion stained). FIG. 7C is an analysis of tissue microarray stained with an a-palladin antibody (histological subtype vs. proportion stained). FIG. 7D is an analysis of tissue microarray stained with an a-palladin antibody (sample type vs. stain intensity). FIG. 7E is an analysis of tissue microarray stained with an a-palladin antibody (WHO grade vs. stain intensity). FIG. 7F is an analysis of tissue microarray stained with an a-palladin antibody (histological subtype vs. stain intensity).

FIG. 8A shows representative ×20 images of the tissue microarray from FIGS. 7A-7F. FIG. 8B shows representative ×63 images of a-palladin antibody-stained murine tumor and normal brain tissue.

FIG. 9A shows an analysis of palladin expression in oligodendroglioma, astrocytoma, and glioblastoma tumors. FIG. 9B shows an analysis of palladin expression in complete response, partial response, stable disease, progressive disease scenarios. FIG. 9C shows a one-way ANOVA with Tukey multiple comparison tests. FIG. 9D is a Pearson correlation test, showing a simple regression line in red with 95% confidence interval, showing time until new tumor following initial treatment.

FIG. 10A shows the correlation of palladin expression with patient age at diagnosis. FIG. 10B shows the correlation of palladin expression with patient age and Karnofsky performance score (KPS). FIG. 10C shows the correlation of palladin expression with patient age and TP53 expression. FIGS. 10A-C together show that palladin is a diagnostic and prognostic marker of glioma tumors. FIG. 10D shows an analysis of palladin expression with respect to MGMT promoter status. FIG. 10E shows an analysis of palladin expression with respect to chromosome 1p/19q codeletion status. FIG. 10F shows an analysis of palladin expression with respect to IDH1 deletion status. FIG. 10G shows palladin transcription levels in normal brain tissue (NBT) and glioma tumors ranging from grades 1-4. FIG. 10H also shows palladin transcription levels in glioma tumors and NBT ranging from grades 1-4.

FIG. 11A shows t-distributed stochastic neighbor embedding (t-SNE) scatter plots of single cell RNA sequencing (scRNAseq) astrocytoma data shaded according to cell type. FIG. 11B shows t-distributed stochastic neighbor embedding (t-SNE) scatter plots of single cell RNA sequencing (scRNAseq) astrocytoma data shaded according to origin of data. FIG. 11C shows t-distributed stochastic neighbor embedding (t-SNE) scatter plots of single cell RNA sequencing (scRNAseq) astrocytoma data shaded according to palladin expression. FIG. 11D show t-SNE scatter plots of scRNAseq glioblastoma multiforme (GBM) data colored according to origin of data, cell type, and palladin expression. FIG. 11E show t-SNE scatter plots of scRNAseq glioblastoma multiforme (GBM) data colored according to palladin expression. FIG. 11F show expression level data according to cell type.

FIG. 12A shows expression quantification of 100 genes similar to Palladin in a number of cell types in astrocytoma and GBM scRNAseq data. FIG. 12B also shows expression quantification in various genes similar to Palladin in a number of cell types in astrocytoma and GBM scRNAseq data.

FIG. 13A shows in-silico flow cytometry of grades 2-3 IDH1-mutant astrocytoma tumors for malignant astrocytes. FIG. 13B shows in-silico flow cytometry of grades 2-3 IDH1-mutant astrocytoma tumors for oligodendrocytes. FIG. 13C shows in-silico flow cytometry of grades 2-3 IDH1-mutant astrocytoma tumors for T-cells. FIG. 13D shows in-silico flow cytometry of grades 2-3 IDH1-mutant astrocytoma tumors for microglia/macrophages.

FIG. 14A shows a gene Ontology enrichment analysis of genes significantly co-expressed with palladin in astrocytoma datasets. Many motility-related terms are observed. FIG. 14B shows a gene Ontology enrichment analysis of genes significantly co-expressed with palladin in glioblastoma multiforme datasets. Many motility-related terms are observed.

### DETAILED DESCRIPTION

The present invention provides methods and compositions for diagnosing and treating cancer in a subject. More particularly, the compositions and methods of the present invention are particularly useful for inhibiting and even preventing cancer metastasis. In some embodiments, the compositions and methods utilize specific compounds such as miRNAs. Such compositions and methods are particularly useful for treating cancer and cancer metastasis, as exemplified herein.

### Definitions

To facilitate an understanding of the present invention, a number of terms and phrases are defined below. It is to be understood that these terms and phrases are for the purpose of description and not of limitation, such that the terminology or phraseology of the present specification is to be interpreted by the skilled artisan in light of the teachings and guidance presented herein, in combination with the knowledge of one of ordinary skill in the art.

As used herein, the term “about”, when referring to a measurable value is meant to encompass variations of +/−10, more preferably +1-5%, even more preferably +/−1, and still more preferably +/−0.1% from the specified value.

The term “cancer” refers to a class of diseases or conditions in which abnormal cells divide without control and can invade nearby tissues. A malignant cancer is one in which a group of tumor cells display one or more of uncontrolled growth (e.g., division beyond normal limits), invasion (e.g., intrusion on and destruction of adjacent tissues), and metastasis (e.g., spread to other locations in the body via lymph or blood). As used herein, the term “metastasize” refers to the spread of cancer from one part of the body to another. A tumor formed by cells that have spread is called a “metastatic tumor” or a “metastasis.” The metastatic tumor contains cells that are like those in the original (primary) tumor. A “cancer cell” or “tumor cell” refers to an individual cell of a cancerous growth or tissue. A tumor refers generally to a swelling or lesion formed by an abnormal growth of cells, which may be benign, pre-malignant, or malignant. Most cancers form tumors, but some, e.g., leukemia, and some blood cancers, do not necessarily form tumors. For those cancers that form tumors, the terms cancer (cell) and tumor (cell) are used interchangeably. The amount of a tumor in an individual is the “tumor burden” which can be measured as the number, volume, or weight of the tumor.

The term “binding” or “stable binding” refers to an association between two substances or molecules, such as the hybridization of one nucleic acid molecule to another (or itself), the association of an antibody with a peptide, or the association of a protein with another protein or nucleic acid molecule. An oligonucleotide molecule (e.g., miRNA, siRNA, or the like) binds or stably binds to a target nucleic acid molecule if a sufficient amount of the oligonucleotide molecule forms base pairs or is hybridized to its target nucleic acid molecule, to permit detection of that binding. Similarly, an antibody binds or stably binds a target protein (e.g., palladian protein target) when a sufficient amount of the antibody binds to its target protein, to permit detection of that binding.

The term “downregulation” refers to the process by which a cell decreases the production and quantities of its cellular components (e.g., Palladin), including RNA and proteins, in response to an external stimulus. The complementary process that involves increase in quantities of cellular components is called upregulation. An example of downregulation is the cellular decrease in the expression of a specific receptor in response to its increased activation by a molecule, such as a hormone or neurotransmitter, which reduces the cell's sensitivity to the molecule. This is an example of a locally acting (negative feedback) mechanism.

As used herein, the term “vector” refers to an expression vector containing a nucleic acid sequence coding for at least part of a gene product capable of being expressed in a host cell. Expression vectors typically contain a variety of “control sequences,” which refer to nucleic acid sequences necessary, for example, for the transcription of an operably linked coding or non-coding sequence in a particular host organism. In addition to control sequences, vectors and expression vectors may contain nucleic acid sequences that serve other functions as well. In some embodiments, an expression vector can be used to encode for or express one or more miRNA molecules in a target cell.

The terms “microRNA” and “miRNA” are directed to a small non-coding RNA molecule that can function in transcriptional and post-transcriptional regulation of target gene expression. The terms encompasses a mature miRNA sequence or a precursor miRNA sequence, including a primary transcript (pri-miRNA) and a stem-loop precursor (pre-miRNA). The biogenesis of a miRNA initiates in the nucleus by RNA polymerase II transcription, generating a primary transcript (pri-miRNA). The primary transcript is cleaved by Drosha ribonuclease III enzyme to produce an approximately 70 nt stem-loop precursor miRNA (pre-miRNA). The pre-miRNA is then actively exported to the cytoplasm where it is cleaved by Dicer ribonuclease to form the mature miRNA. One strand of this miRNA is incorporated into an RNA-induced silencing complex (RISC) which recognizes target mRNAs through imperfect base pairing with the miRNA, and most commonly results in translational inhibition or destabilization of the target mRNA. Typically, the target mRNA contains a sequence complementary to a “seed” sequence of the miRNA, which usually corresponds to nucleotides 2-8 of the miRNA. The seed sequence is considered to be essential for the binding of the miRNA to the mRNA. Information concerning miRNAs and associated pri-miRNA and pre-miRNA sequences is available in miRNA databases such as miRBase (Griffiths-Jones et al. 2008 Nucl Acids Res 36, (Database Issue: D154-D158) and the NCBI human genome database.

The terms “miRNA molecules” and “miR molecules” refer to the miR-96 and/or miR-182 miRNA molecules. When referring to the miR molecules, the reference is to either one or both of said miRNA molecules. Each possibility being a separate embodiment.

As used herein, the terms “metastasis”, “cancer metastasis” or “tumor metastasis” are used interchangeably and refer to the growth of cancerous cells derived from the primary cancerous tumor in another location or tissue. Metastasis also encompasses micrometastasis, which is the presence of an undetectable amount of cancerous cells in an organ or body part which is not directly connected to the organ of the original, primary cancerous tumor. Metastasis can also be defined as several steps of a process, such as the departure of cancer cells from an original tumor site, and migration and/or invasion of cancer cells to other parts of the body.

As referred to herein, the terms “nucleic acid”, “nucleic acid molecules” “oligonucleotide”, “polynucleotide”, and “nucleotide” may interchangeably be used herein. The terms are directed to polymers of deoxyribonucleotides (DNA), ribonucleotides (RNA), and modified forms thereof in the form of a separate fragment or as a component of a larger construct, linear or branched, single stranded, double stranded, triple stranded, or hybrids thereof. The term also encompasses RNA/DNA hybrids. The polynucleotides may include sense and antisense oligonucleotide or polynucleotide sequences of DNA or RNA. The DNA or RNA molecules may be, for example, but not limited to: complementary DNA (cDNA), genomic DNA, synthesized DNA, recombinant DNA, or a hybrid thereof or an RNA molecule such as, for example, mRNA, shRNA, siRNA, miRNA, Antisense RNA, and the like. Each possibility is a separate embodiment. The terms further include oligonucleotides composed of naturally occurring bases, sugars, and covalent internucleoside linkages, as well as oligonucleotides having non-naturally occurring portions, which function similarly to respective naturally occurring portions.

The term “Palladin” refers to a protein that in humans is encoded by the PALLD gene. Palladin is a component of actin-containing microfilaments that control cell shape, adhesion, and contraction. Palladin is a part of the myotilin-myopalladin-palladin family and plays an important role in modulating the actin cytoskeleton. Palladin, in contrast to myotilin and myopalladin, which are expressed only in striated muscle, is expressed ubiquitously in cells of mesenchymal origin. In humans, in seven different isoforms exist, some of which arise through alternative splicing. In mice, three major isoforms of palladin arise from a single gene. These isoforms contain between three and five copies (depending on the isoform) of an Ig-like domain and between one and two copies of a polyproline domain.

The terms “polypeptide,” “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The term “construct”, as used herein, refers to an artificially assembled or isolated nucleic acid molecule which may include one or more nucleic acid sequences, wherein the nucleic acid sequences may include coding sequences (that is, sequence which encodes an end product), regulatory sequences, non-coding sequences, or any combination thereof. The term construct includes, for example, vector but should not be seen as being limited thereto.

The term “plasma” refers to the liquid part of the blood and lymphatic fluid, which makes up about half of the volume of blood. Plasma is devoid of cells and, unlike serum, has not clotted. Blood plasma contains antibodies and other proteins. It is taken from donors and made into medications for a variety of blood-related conditions.

As used herein the term “vector” refers to recombinant constructs engineered to encode or express polynucleotides in a target cells, such as DNA, RNA, miRNA, shRNA, siRNA, antisense oligonucleotides, and the like. Vectors may include such vectors as, but not limited to, viral and non-viral vectors, plasmids, and the like.

The term “treating” and “treatment” as used herein refers to abrogating, inhibiting, slowing or reversing the progression of a disease or condition, ameliorating clinical symptoms of a disease or condition or preventing the appearance of clinical symptoms of a disease or condition. The term “preventing” is defined herein as barring a subject from acquiring a disorder or disease or condition.

The term “treating brain tumors” is directed to include one or more of the following: a decrease in the rate of growth of the brain tumor (e.g. the brain tumor still grows but at a slower rate); cessation of growth of the cancerous growth, e.g., stasis of the brain tumor growth, and, the tumor diminishes or is reduced in size. The term also includes reduction in the number of metastases, reduction in the number of new metastases formed, slowing of the progression of cancer from one stage to the other and a decrease in the angiogenesis induced by the cancer. In most preferred cases, the tumor is totally eliminated. Additionally included in this term is lengthening of the survival period of the subject undergoing treatment, lengthening the time of diseases progression, tumor regression, and the like. In some embodiments, the cancer is a solid tumor. In some exemplary embodiments, the cancer is breast cancer.

The term “siRNA” is directed to a double stranded nucleic acid molecule capable of RNA interference or “RNAi.” (See, for example, Bass Nature 411:428-429, 2001; Elbashir et al., Nature 411:494-498, 2001; and Kreutzer et al., International PCT Publication No. WO 00/44895; Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914.) As used herein, siRNA molecules need not be limited to those molecules containing only RNA, but further encompasses chemically modified nucleotides and non-nucleotides having RNAi capacity or activity. In an example, a siRNA molecule is one that reduces or interferes with the biological activity of palladin or inhibits its binding to the actin cytoskeleton.

As used herein the term “subject” is interchangeable with an individual or patient. According to some embodiments, the subject is a mammal. According to some embodiments, the subject is a human. According to some embodiments, the subject is symptomatic. According to other embodiments, the subject is asymptomatic. In some embodiments, the subject is a human afflicted with cancer. In some embodiments, the subject is preferably an individual with the C allele of the polymorphic site within the PALLD gene identified by reference number rs1071738. In some embodiments, the subject is at risk of developing metastasis. In other embodiments, the subject has already developed metastases.

In some embodiments, a method of treating brain tumors in a subject is disclosed. In embodiments, detecting or diagnosing glioma in the subject includes: a) measuring PALLD mRNA levels in a plasma sample of the subject; correlating the PALLD mRNA levels to PALLD mRNA plasma levels corresponding to a likelihood of having a specific type of glioma; making a diagnosis based on the PALLD mRNA levels of the plasma sample as compared to the expected PALLD mRNA plasma levels corresponding to a likelihood of having a specific type of glioma; and b) treating subject diagnosed with specific type of glioma or having the likelihood of developing specific type of glioma with at least one therapeutic agent for treating glioma. In other embodiments, disclosed is a method of treating brain tumors wherein the at least one therapeutic agent (e.g., miR-96 and miR-182, or the like) causes downregulation of palladin within targeted tissue of a subject. In yet another embodiment, the at least one therapeutic agent causing downregulation of palladin comprises miR-96 and miR-182, wherein the miR-96 and miR-182 are bound to gold nanoparticles and/or and are further embedded in a hydrogel. “measuring PALLD mRNA levels”.

In other embodiments, diagnosis is made based on immunohistological staining of palladin. In particular, results may be validated using IHC staining of tumor samples together with qRT-PCR of glioma cell lines. For example, palladin may be stained using BOND-III (Leica Biosystems). In one method contemplated by the present invention, post primary antibodies and polymer are combined and incubated for 1 hour one at a time. Then, 3,3′-diaminobenzidine (DAB) is added followed by hematoxylin counterstain. The slide is then analyzed by a pathologist. In embodiments, all incubation steps may be carried out at room temperature using reagents in standard supplied with BOND-III. See the methods section below for further detail. In another embodiment, the therapeutic agent includes at least one miRNA molecule selected from the group consisting of miR-96 and miR-182, or at least one vector expressing or encoding the same, for use in reducing or preventing the specific type of glioma. In other embodiments, the therapeutic agent is formulated for systemic administration, local administration, intra-tumor administration, enteral administration, oral administration, sublingual and buccal administration, rectal administration, intravenous administration, intramuscular administration, and/or subcutaneous administration.

In some embodiments, the glioma comprises a cancer associated with abnormal palladin expression and/or activity. In other embodiments, the glioma is an ependymoma, the glioma is selected from the group consisting of astrocytoma or oligodendroglioma, or the glioma comprises a glioblastoma (or another high-grade glioma originating in astrocytes). In other embodiments, the subject is at risk of developing metastasis and the administering is carried out prior to metastasis formation. In yet another embodiment, the subject has already developed metastases and the administering is carried out after metastasis formation.

In other embodiments, a method of treating brain tumors is disclosed wherein a therapeutic agent inhibits the binding of palladin to actin cytoskeleton (e.g., actin binding proteins, Jasplakinolide, Latrunculin B, and the like). In embodiments, over 150 actin-binding proteins (ABPs) are known that may influence localization, polymerization dynamics, crosslinking, and organization of actin. These ABPs have various means of regulating actin, which include the ability to sequester monomeric actin (G-actin), nucleate filament formation, sever filamentous actin (F-actin), generate branched arrays of actin, and cap actin filaments.

In embodiments, glioblastoma tumors express approximately 150% and 70% more palladin than oligodendroglioma, and astrocytoma tumors, respectively (P<0.0001 and P<0.0001). In other embodiments, glioblastoma tumors express approximately (100%, 125%, 150%, 175%, or 200%) and (50%, 70%, 90%, 100%, 140%) more palladin than oligodendroglioma, and astrocytoma tumors, respectively (P<0.0001 and P<0.0001).

In still other embodiments, a method of detecting or diagnosing glioma tumor type in a subject is disclosed. Said method includes the steps of measuring PALLD mRNA levels in a plasma sample of the subject; correlating the PALLD mRNA levels to PALLD mRNA plasma levels corresponding to a likelihood of having a specific type of glioma; and making a diagnosis based on the PALLD mRNA levels of the plasma sample as compared to the expected PALLD mRNA plasma levels corresponding to a likelihood of having a specific type of glioma. In other embodiments, said method of detecting or diagnosing glioma comprises an adult-type diffuse glioma. In another embodiment, a method is disclosed delineating brain tumor tissue from surrounding brain, the method comprising injecting a palladin-associating fluorophore or prodrug comprising a palladin-associating fluorophore into a subject for fluorescence-guided neurosurgery.

Further to the above, the methods of the present invention may be used to diagnose any type of brain cancer but are particularly useful for diagnosing gliomas. In embodiments, blood plasma diagnostic measurements are directed to palladin protein levels. The methods of the present invention may be used to detect palladin protein in any type of sample from a patient with suspected brain cancer. In embodiments, the methods are particularly useful for detecting palladin protein in blood samples, cerebrospinal fluid (CSF) samples, and brain tissue samples. The methods of the present invention may use any suitable method to detect palladin. Suitable methods for detecting palladin include, but are not limited to, immunoassays, such as enzyme-linked immunosorbent assays (ELISAs) and Western blots; mass spectrometry; and gene expression profiling.

In embodiments, the predetermined threshold for palladin that is used to diagnose brain cancer, particularly a glioma, may be determined by a variety of methods. One method for determining the predetermined threshold is to compare the level of palladin in a group of patients with brain cancer, particularly gliomas, to the level of palladin in a group of healthy patients. The predetermined threshold may be set at a level that is above the level of palladin in the group of healthy patients. Another method for determining the predetermined threshold is to use a receiver operating characteristic (ROC) curve. An ROC curve is a graph that shows the sensitivity and specificity of a diagnostic test at different cutoff points. The predetermined threshold may be set at a cutoff point that corresponds to a desired level of sensitivity and specificity.

In one example, a blood sample is obtained from a patient suspected of having a glioma. The level of palladin in the blood sample is detected using an ELISA. The level of palladin is above the predetermined threshold, and the patient is diagnosed with a glioma. In another example, a CSF sample is obtained from a patient suspected of having a glioma. The level of palladin in the CSF sample is detected using a mass spectrometer. The level of palladin is above the predetermined threshold, and the patient is diagnosed with a glioma. In yet another example, a brain tissue sample is obtained from a patient suspected of having a glioma. The level of palladin in the brain tissue sample is detected using gene expression profiling. The level of palladin is above the predetermined threshold, and the patient is diagnosed with a glioma.

As described above, a method of treating brain tumors is herein disclosed wherein a therapeutic agent causes downregulation of palladin. In embodiments, said agent comprises miR-96/miR-182, bound to gold nanoparticles and embedded in a hydrogel. Notably, in some embodiments SRC-mediated, platelet-derived growth factor-induced membrane ruffling and lamellipodia formation required both palladin and SPIN90. Taken together, it is reasonable to assume that downregulation of palladin or inhibition of its binding to the actin cytoskeleton might curb the aggressive phenotype of glioma tumors.

In some embodiments, there is provided a method for treating brain cancer in a subject in need thereof, the method comprising administering to the subject at least one miRNA molecule selected from the group consisting of miR-96 and miR-182, or at least one vector expressing or encoding the same, thereby treating brain cancer in the subject. In some embodiments, there is provided a method for treating brain cancer in a subject in need thereof, the method comprising administering to the subject miR-96 and/or miR-182, or a corresponding vector expressing or encoding the same, thereby treating brain cancer in the subject.

In some embodiments, there is provided a method of treating brain cancer in a subject in need thereof, the method including inhibiting or reducing expression of palladin in the brain cancer cells. In some embodiments, reducing or inhibiting expression of palladin is achieved by administration of miR-96 and/or -miR-182 or one or more vectors encoding for said miRNA molecules. Each possibility is a separate embodiment. In some embodiments, the methods may further include determining that the subject is carrying the C allele of the single nucleotide polymorphism (SNP) rs1071738 prior to administering the at least one miRNA molecule or the at least one vector expressing or encoding the same.

In some embodiments, the subject is at risk of developing metastasis and the administering is carried out prior to metastasis formation. In some embodiments, the subject has already developed metastases and the administering is carried out after metastasis formation. In some embodiments, the brain cancer is a brain cancer associated with an abnormal palladin expression and/or activity. In some embodiments, there is provided a pharmaceutical composition comprising at least one miRNA molecule selected from the group consisting of miR-96 and miR-182, or at least one vector expressing or encoding the same, for use in reducing or preventing brain cancer metastasis. In some embodiments, there is provided a pharmaceutical composition comprising miR-96 and/or miR-182, or corresponding vector(s) expressing or encoding the same, for use in reducing or preventing brain cancer metastasis.

In some embodiments, there is provided a pharmaceutical composition comprising at least one miRNA molecule selected from the group consisting of miR-96 and miR-182, or at least one vector expressing or encoding the same, for use in treating brain cancer. In some embodiments, there is provided a pharmaceutical composition comprising miR-96 and/or miR-182, or corresponding vector(s) expressing or encoding the same, for use in treating brain cancer.

In some embodiments, there is provided a pharmaceutical composition comprising miR-96 and/or miR-182, or corresponding vector(s) expressing or encoding the same, for use in reducing palladin expression in cancer cells. In some embodiments, each of miR-96 and miR-182, or the corresponding vectors expressing or encoding the same are formulated in distinct compositions (such as pharmaceutical compositions), that may be administered concomitantly or separately. In some embodiments, both the miR-96 and the miR-182, or the corresponding vectors expressing or encoding the same are formulated in one composition (such as pharmaceutical composition).

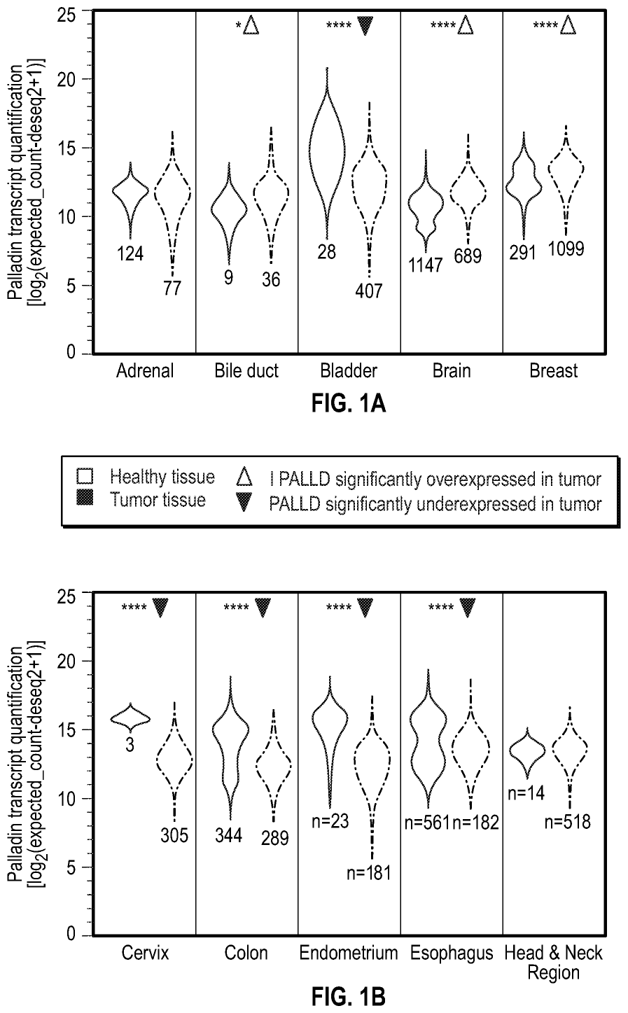

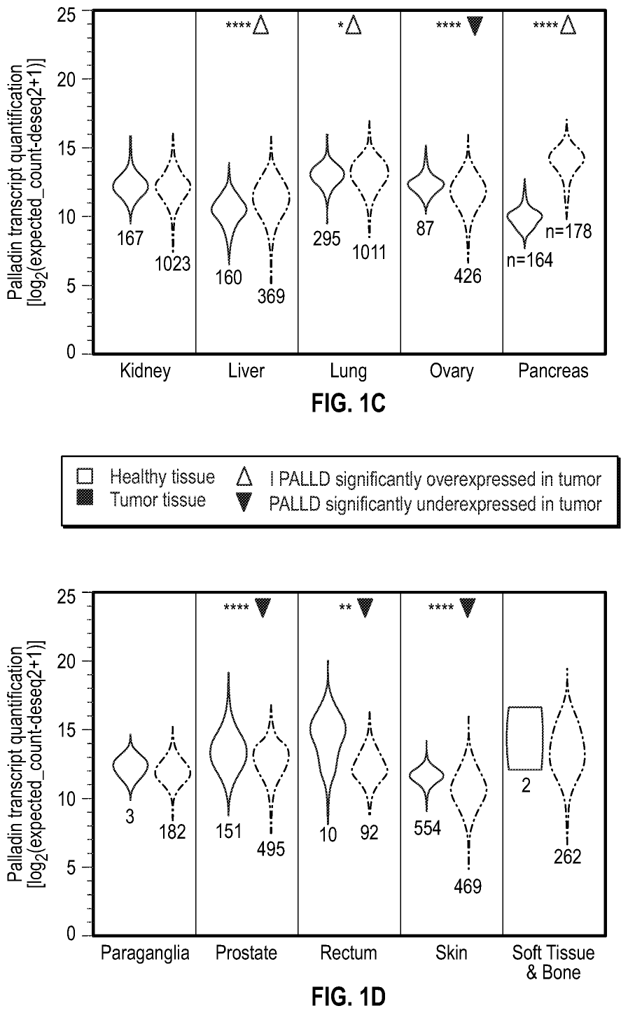

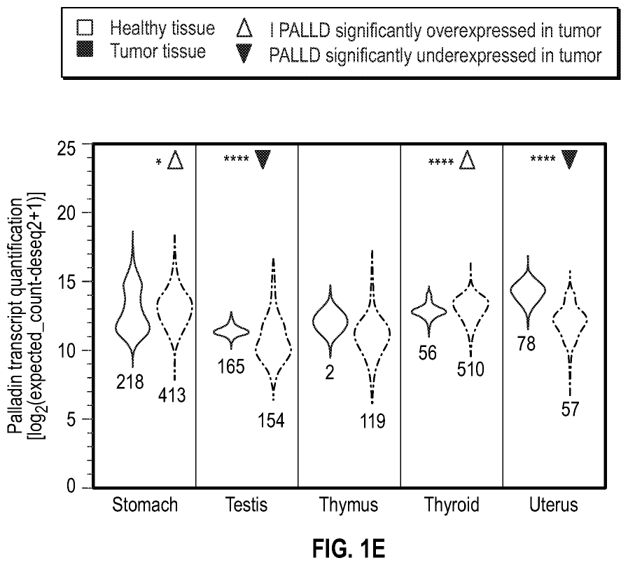

In some embodiments, palladin is overexpressed in adult glioma tumors and is correlated with shorter overall survival. In embodiments, transcription of the PALLD gene was analyzed in tumorous and healthy tissues. In other embodiments, expression data from 25 organs were acquired from the TCGA (tumor samples) and GTEx (healthy tissue samples) datasets; the sample sizes ranged from two to 1098 per organ after outlier removal. In some embodiments, eight tumor types were identified with significant PALLD overexpression relative to healthy tissue: bile duct, brain, breast, liver, lung, pancreas, stomach, and thyroid (See FIGS. 1A-1E, Table 1). In embodiments, tumors in eleven organs exhibited significant downregulation: the bladder, cervix, colon, endometrium, esophagus, ovary, prostate, rectum, skin, testis, and uterus. In other embodiments, tumors from the remaining body regions showed no significant difference in expression between cancer and normal tissue.

FIG. 1A shows palladin expression in healthy versus tumor samples, including tumor samples from adrenal, bile duct, bladder, brain, and breast cancers. FIG. 1B shows palladin expression in healthy versus tumor samples, including tumor samples from cervix, colon, endometrium, esophagus, and head/neck cancers. FIG. 1C shows palladin expression in healthy versus tumor samples, including tumor samples from kidney, liver, lung, ovary, and pancreas cancers. FIG. 1D shows palladin expression in healthy versus tumor samples, including tumor samples from paraganglia, prostate, rectum, skin, and soft tissue/bone cancers. FIG. 1E shows palladin expression in healthy versus tumor samples, including tumor samples from stomach, testis, thymus, thyroid, and uterus cancers. In summary, FIGS. 1A-1E show that wild-type palladin-4 mRNA is overexpressed in adult gliomas and is correlated with decreased survival.

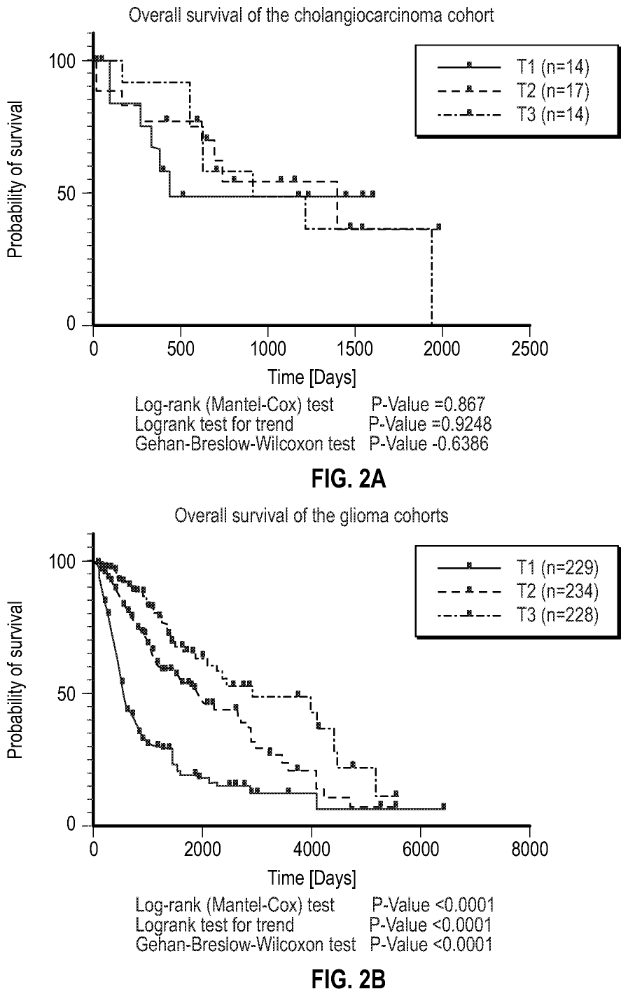

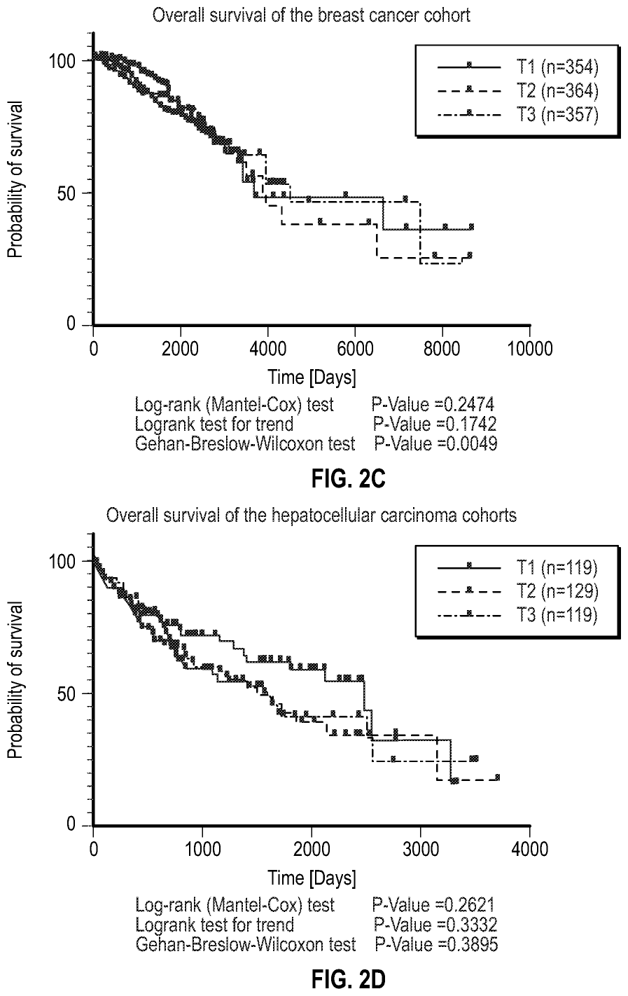

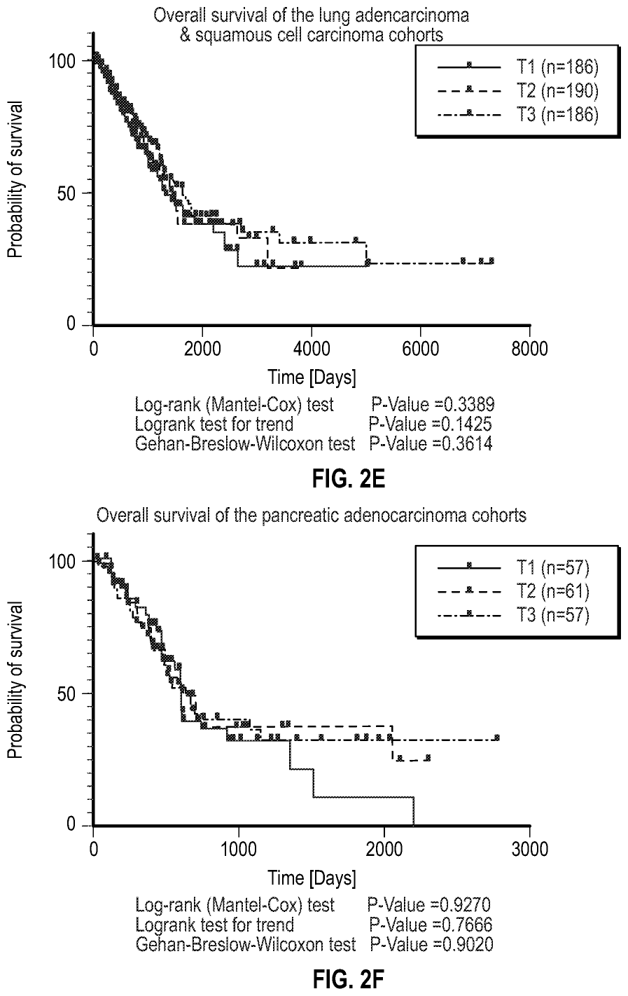

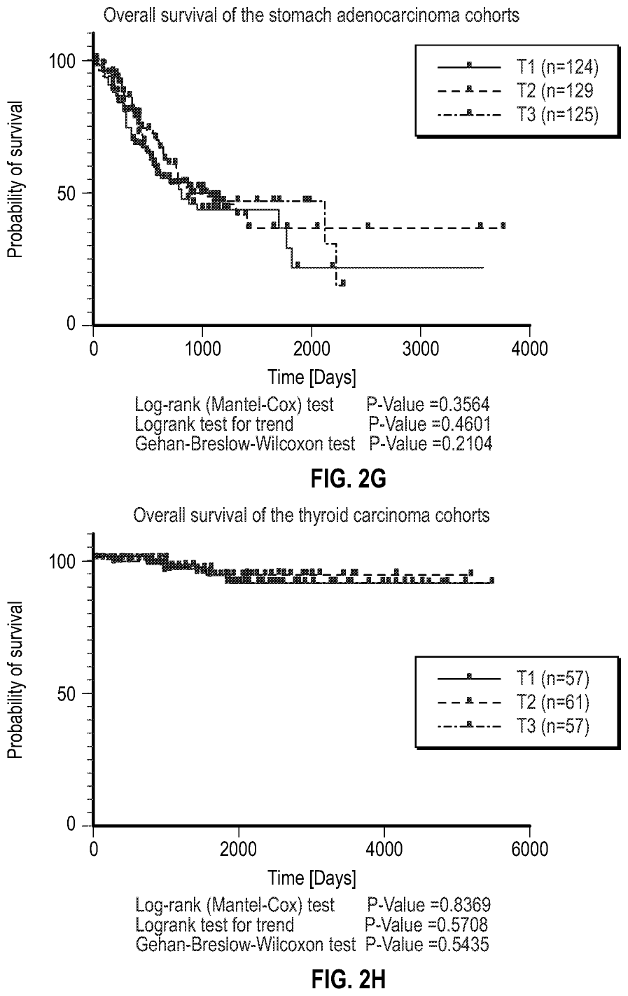

According to the present disclosure, palladin has a tumor promoting function. Therefore, palladin overexpressing tumors were targeted for investigation into the suitability of palladin as a marker for predicting disease incidence, progression, and prognosis as well as a therapeutic target. Overall survival data in palladin overexpressing tumors was analyzed. Survival data from each of the eight palladin-overexpressing TCGA cohorts were stratified into three tertiles based on palladin expression levels, and analyzed. Significant differences in survival probability between high, moderate, and low palladin-expressing groups were only observed in brain tumors (combined glioma cohort comprised by the low-grade glioma (LGG) and GBM sub-cohorts) (χ2Mental-Cox=115.7, df=2, P<0.0001, FIGS. 2A-2H). The differences in survival mimics a dose response relation; median survival was 548, 1886, and 2907 days in tumors with high, medium, and low levels of palladin, respectively. To further validate the results, patient survival curves were analyzed with respect to palladin expression of all the adult glioma datasets available on the GlioVis website, after determining the optimal cutoff. In embodiments, of the 17 datasets that included both survival and palladin expression data, 10 exhibit a significant decrease in survival in palladin-overexpressing samples and one showed a similar non-significant trend. The 6 remaining datasets exhibited 4 significant and 2 non-significant trends of increased survival time in palladin-overexpressing samples.

To account for the presence of normal, tumor-adjacent tissue (NAT) in the TCGA cohorts, the TCGA-GBMLGG dataset was reanalyzed with respect to the origin of the sample. Analysis of all 1130 samples (HKruskal-Wallis=275.5, P<0.0001) revealed that, compared to healthy donor brain tissue, both primary and recurrent glioma tumors expressed roughly twice as much palladin (P<0.0001 and P<0.0001, respectively) and NAT (P=0.0052 and P=0.0042, respectively). In embodiments, overexpression of PALLD in glioma tumors compared to non-tumor samples was also validated in six of the seven datasets containing non-tumor samples in the GlioVis website.

TABLE 1
Summary of all the comparisons examined of palladin expression
in tumor and healthy tissues from different organs.

| | | | | PALLD |
|---|---|---|---|---|
| Organ/Region | Study & Cohort | Statistic | P | in Tumor |
| Adrenal gland | TCGA-ACC + GTEx | U_{Mann-Whitney} = 4299 | 0.2364 | → |
| Bile duct | TCGA-CHOL + GTEx | t_{unpaired} = 2.242, df = 43 | 0.0302 | ↑ |
| Bladder | TCGA-BLCA + GTEx | t_{unpaired} = 8.511, df = 433 | <0.0001 | ↓ |
| Brain | TCGA-GBM + TCGA-LGG + GTEx | U_{Mann-Whitney} = 147235 | <0.0001 | ↑ |
| Breast | TCGA-BRCA + GTEx | U_{Mann-Whitney} = 110310 | <0.0001 | ↑ |
| Cervix | TCGA-CESC + GTEx | t_{unpaired} = 5.24, df = 306 | <0.0001 | ↓ |
| Colon | TCGA-COAD + GTEx | U_{Mann-Whitney} = 21873 | <0.0001 | ↓ |
| Endometrium | TCGA-UCEC + GTEx | U_{Mann-Whitney} = 308 | <0.0001 | ↓ |
| Esophagus | TCGA-ESCA + GTEx | U_{Mann-Whitney} = 48610 | <0.0001 | ↓ |
| Head & neck | TCGA-HNSC + GTEx | U_{Mann-Whitney} = 10902 | 0.6338 | → |
| reion | | | | |
| Kidney | TCGA-KIRC + TCGA-KIRP + GTEx | U_{Mann-Whitney} = 81033 | 0.2866 | → |
| Liver | TCGA-LIHC + GTEx | U_{Mann-Whitney} = 14504 | <0.0001 | ↑ |
| Lung | TCGA-LUAD + TCGA-LUSC + GTEx | U_{Mann-Whitney} = 182850 | 0.014 | ↑ |
| Ovary | TCGA-OV + GTEx | U_{Mann-Whitney} = 12349 | <0.0001 | ↓ |
| Pancreas | TCGA-PAAD + GTEx | U_{Mann-Whitney} = 17.5 | <0.0001 | ↑ |
| Paraganglia | TCGA-PCPG + GTEx | t_{unpaired} = 0.7294, df = 183 | 0.4667 | → |
| Prostate | TCGA-PRAD + GTEx | U_{Mann-Whitney} = 27055 | <0.0001 | ↓ |
| Rectum | TCGA-READ + GTEx | t_{Welch-corrected} = 4.435, | 0.0013 | ↓ |
| | | df = 9.872 | | |
| Skin | TCGA-SKCM + GTEx | U_{Mann-Whitney} = 67245 | <0.0001 | ↓ |
| Soft tissue & bone | TCGA-SARC + GTEx | U_{Mann-Whitney} = 215 | 0.6772 | → |
| Stomach | TCGA-STAD + GTEx | U_{Mann-Whitney} = 37554 | 0.0105 | ↑ |
| Testis | TCGA-TGCT + GTEx | U_{Mann-Whitney} = 7660 | <0.0001 | ↓ |
| Thymus | TCGA-THYM + GTEx | U_{Mann-Whitney} = 45.5 | 0.1532 | → |
| Thyroid | TCGA-THCA + GTEx | U_{Mann-Whitney} = 10832 | 0.0028 | ↑ |
| Uterus | TCGA-UCS + TCGA-UCEC + GTEx | t_{Welch-corrected} = 12.95, | <0.0001 | ↓ |
| | | df = 85.67 | | |

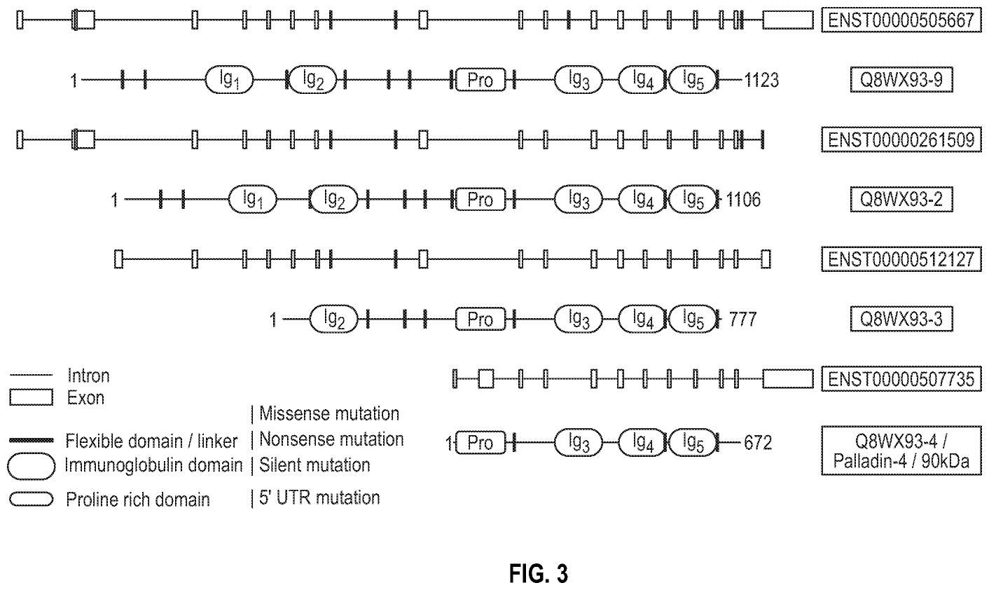

In embodiments, palladin isoform 4 is specifically overexpressed in adult glioma tumors. To investigate the origin of palladin's overexpression in glioma tumors, data of various transcript expression levels in 1830 healthy and tumor samples were analyzed. Overall, it was determined that the PALLD gene has 18 transcripts, of which five are non-coding. Of the remaining 13, only four contain complete reading frames (FIG. 3). In embodiments, from analysis of the transcription patterns, it was concluded that ENST00000505667, the canonical Ensembl palladin transcript, was not expressed in healthy brain tissue or in LGG or HGG tumors (HKruskal-Wallis=167.6, P<0.0001). ENST00000261509 (HKruskal-Wallis=537.3, P<0.0001) doubled its transcription in LGG and HGG tumors compared to normal tissue (P<0.0001 and P<0.0001, respectively). A slight decrease in expression was seen from the LGG to HGG (P=0.0283). ENST00000512127 (HKruskal-Wallis=804.3, P<0.0001) was not expressed in healthy brains or LGGs but was significantly more expressed in HGGs (P<0.0001 and P<0.0001, respectively). ENST00000507735 (HKruskal-Wallis=432.7, P<0.0001), also known as palladin 4 or the 90 kDa isoform, nearly doubled its expression from healthy tissue to LGG tumors, and doubled again from LGG to HGG tumors (P<0.0001 and P<0.0001, respectively).

In embodiments, somatic mutations in palladin's genomic sequence were found to be extremely rare. To assess the prevalence and characteristics of somatic mutations in the PALLD gene in glioma tumors, the TCGA-GBMLGG dataset was analyzed. Of the 1154 samples in the dataset, 826 had data on genomic variation. FIG. 3 shows a schematic representation of palladin's complete coding transcripts, their protein products, and somatic mutations. Mutations in palladin's genomic sequence were identified in only six samples (FIG. 3). In embodiments, these mutations included one 5′ untranslated region (UTR) mutation, two in-frame missense mutations, and one premature stop codon insertion. Two other tumors had a mixture of silent and missense mutations. In embodiments, due to the low prevalence of palladin mutations in gliomas, the impact of mutations in specific domains or the presence of mutations in general could not be determined.

In some embodiments, palladin is not overexpressed in pediatric glioma tumors. Expanding the investigation into palladin's role in glioma tumors, pediatric glioma datasets from the GlioVis website were analyzed. In embodiments, nine of the 24 datasets available contained relevant data and were used. Ultimately, differences tying palladin to any specific clinical feature were not observed. Differences in palladin expression between fetal and adult human brains has been previously documented. Eight fetal and eight adult brain samples were obtained from the frontal lobe and cerebellum of donors. An expression analysis of samples revealed significantly higher average PALLD expression in fetal than in adult brains (t-unpaired=5.042, df=14, P=0.0002).

In embodiments, palladin expression is confined predominantly to the cancerous tissue in the brain. To validate the findings total RNA was isolated from several mouse glioblastoma (GBM) cell lines and normal brain tissue (NBT). Quantitative real-time PCR (qRT-PCR) showed overexpression of palladin in GBM cell lines compared to NBT (F=235.3, P<0.0001, FIGS. 1A-1E). Multi-tissue arrays were obtained with over 200 tissue cores of various CNS pathologies. Slides were immunohistochemically (IHC) stained with an anti-palladin antibody. Survey of the slide elucidated palladin's presence in cell membranes, the cytoplasm, and the nuclei in the CNS. Healthy brain tissue and NAT exhibited weak to no staining in all three cellular compartments (FIGS. 2B-C). Benign tumors exhibited weak to moderate membrane and cytoplasmic staining in all surveyed cells and in the nuclei of 25% of the cells. In cores featuring glial hyperplasia, weak to moderate staining exclusively in the nucleus was seen in 75% of the cells. Weak to moderate staining in all three compartments was identified in 25% or less of the cells in CNS inflammation tissue cores. Malignant tumors exhibited weak to moderate membrane and cytoplasmic staining in 75% and 50% of cells, respectively. In approximately 15% of the malignant tumor cells, weak nuclear staining was observed.

FIG. 2A shows overall survival of the cholangiocarcinoma cohort. FIG. 2B shows overall survival of the glioma cohorts. FIG. 2C shows overall survival of the breast cancer cohorts. FIG. 2D shows overall survival of the hepatocellular carcinoma cohorts. FIG. 2E shows overall survival of the pancreatic adenocarcinoma cohorts. FIG. 2F shows overall survival of the glioma cohorts. FIG. 2G shows overall survival of the stomach adenocarcinoma cohorts. FIG. 2H shows overall survival of the thyroid carcinoma cohorts. In summary, FIGS. 2A-2H shows overall survival of all palladin overexpressing tumors in FIGS. 1A-1E, stratified into three groups based on expression level.

In embodiments, staining was analyzed with respect to WHO CNS tumor grade. Strong nuclear staining was seen almost exclusively in WHO CNS grade 1 tumors. The prevalence of membrane and cytoplasmic staining increased concordantly with tumor grade, but did not surpass intensity levels of moderate and weak, respectively. Weak to moderate nuclear staining was observed in ˜8.5-11.3% of cells in astrocytomas, oligodendrogliomas, and ependymomas. In contrast, weak to moderate membranal staining was seen in over 75% of the cells evaluated in all three tumor subtypes. Finally, weak staining of the cell membrane was observed in most oligodendroglioma and ependymoma tumors, while the same intensity was seen in only 25% of astrocytoma tumors. In the vast majority of the cores, weak staining in the neuropil was seen.

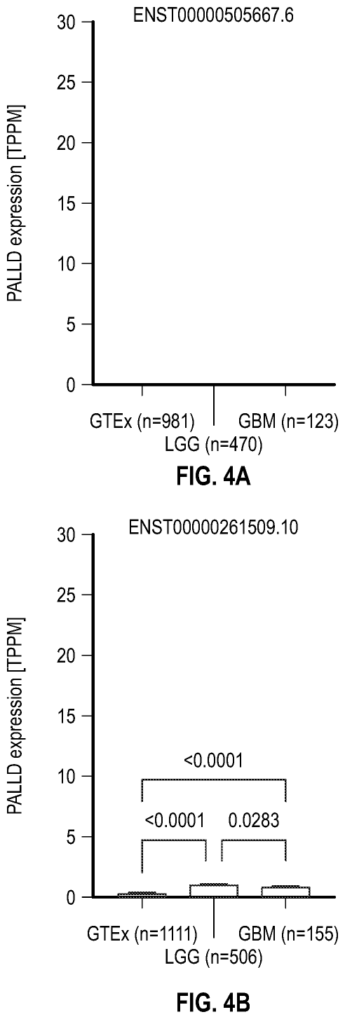

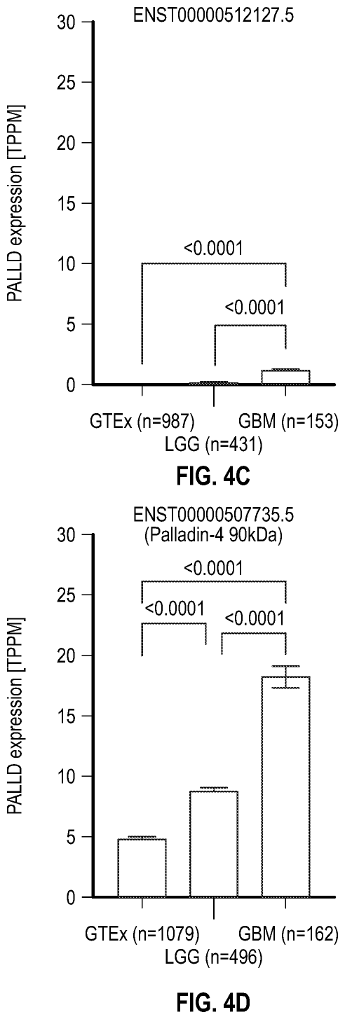

Notably, FIG. 4A shows expression levels the coding palladin isoform ENST00000505667.6. FIG. 4B shows expression levels the coding palladin isoform ENST00000261509.1 FIG. 4C shows expression levels the coding palladin isoform ENST00000512127.5. FIG. 4D shows expression levels the coding palladin isoform ENST00000507735.5.

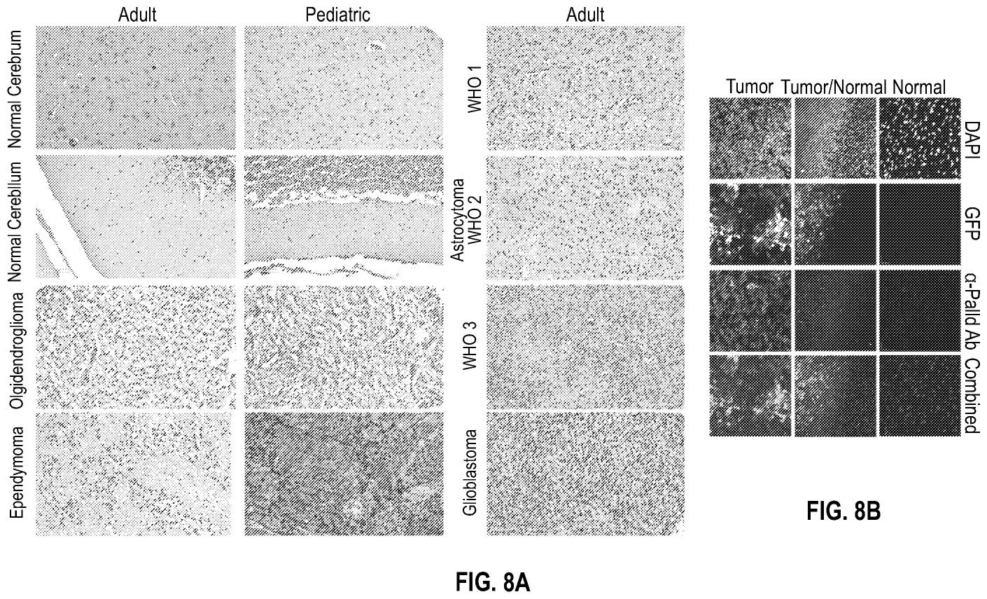

In other embodiments, palladin distribution in the area of contact between the tumor and NBT was inspected. Mouse GBM cells that stably expressed GFP in mice brains were injected. Images showed clear localization of palladin staining to the area of cancer cells and not to the healthy tissue (FIG. 8B lower row and upper row, respectively). In embodiments, the immunofluorescent staining images highlight the localization of palladin to the membrane, but in contrast to the IHC images, they do not show staining in the tumor cell nucleus or cytoplasm. Palladin immunofluorescence staining extends faintly, slightly beyond the boundary layer of labeled tumor cells (FIG. 8B middle row).

In another embodiment, the disclosed method comprises administering to the subject an effective amount of a composition comprising at least three suppressive miRNA. According to yet another embodiment, the method comprises administering to the subject an effective amount of a composition comprising at least four suppressive miRNA. According to yet another embodiment, the method comprises administering to the subject an effective amount of a composition comprising at least one miR-agonist capable of mimicking the activity of at least three suppressive miRNAs. According to one embodiment, the at least one compound is selected from the group consisting of double-stranded RNA, small-interfering RNA, antisense nucleic acid, antagonist of the at least one miRNA and enzymatic RNA molecules.

In embodiments, a method for detecting glioma tumors is disclosed, in which a sample of tissue is obtained from a patient, the expression of palladin is detected in the tissue sample, and it is determined that the expression of palladin is greater in the tissue sample than in a normal tissue sample. In other embodiments, a method for diagnosing glioma tumors is disclosed, in which a sample of tissue is obtained from a patient, the expression of palladin is detected in the tissue sample, and the expression of palladin in the tissue sample is compared to a reference range for palladin expression in normal tissue.

In still other embodiments, a method for monitoring the progression of glioma tumors is disclosed, in which a sample of tissue is obtained from a patient, the expression of palladin is detected in the tissue sample, and the expression of palladin in the tissue sample is compared to a previous measurement of palladin expression in the patient. In yet other embodiments, a method for predicting the response to treatment of glioma tumors is disclosed, in which a sample of tissue is obtained from a patient, the expression of palladin is detected in the tissue sample, and the expression of palladin in the tissue sample is correlated with the response to treatment of the patient.

In some embodiments, kit for detecting glioma tumors is disclosed, in which a palladin antibody is included and instructions are provided for using the antibody to detect the expression of palladin in a tissue sample. In other embodiments, a kit for diagnosing glioma tumors is disclosed, in which a palladin antibody is included and instructions are provided for using the antibody to detect the expression of palladin in a tissue sample. In some embodiments, a kit for monitoring the progression of glioma tumors is disclosed, in which a palladin antibody is included and instructions are provided for using the antibody to detect the expression of palladin in a tissue sample.

In some embodiments, a kit for predicting the response to treatment of glioma tumors is disclosed, in which a palladin antibody is included and instructions are provided for using the antibody to detect the expression of palladin in a tissue sample. In other embodiments, a method for treating glioma tumors is disclosed, in which a pharmaceutical composition comprising a palladin inhibitor is administered to a patient. In embodiments, a pharmaceutical composition for treating glioma tumors is disclosed, in which a palladin inhibitor is included. In other embodiments, a method for preventing the development of glioma tumors is disclosed, in which a pharmaceutical composition comprising a palladin inhibitor is administered to a patient.

Further to the above, in some embodiments, a pharmaceutical composition for preventing the development of glioma tumors is disclosed, in which a palladin inhibitor is included. In some embodiments, a pharmaceutical composition for increasing the survival of a patient with glioma tumors is disclosed, in which a palladin inhibitor is included. In some embodiments, a method for increasing the efficacy of treatment for glioma tumors is disclosed, in which a pharmaceutical composition comprising a palladin inhibitor is administered to the patient. In some embodiments, a pharmaceutical composition for increasing the efficacy of treatment for glioma tumors is disclosed, in which a palladin inhibitor is included.

In embodiments, glioblastoma tumors are characterized by high levels of palladin expression. To further understand the role of palladin role in LGG tumors, 525 non-GBM tumor samples grouped by the dataset's original histopathologic type as astrocytomas, oligoastrocytomas, or oligodendrogliomas were analyzed. PALLD mRNA levels differed significantly between tumor types (HKruskal-Wallis=90.08, P<0.0001). The highest levels of PALLD expression were found in astrocytoma tumors, followed by oligoastrocytoma, and finally, oligodendroglioma tumors.

In other embodiments, the 5-year overall survival of LGG tumors grouped into three tertiles based on palladin expression was analyzed. According to the disclosure, astrocytoma tumors exhibit a significant negative correlation between palladin expression and survival time (χ2Mental-Cox=13.1, df=2, P=0.0014). Median survival in astrocytomas featuring high, medium, and low levels of PALLD expression were 814, 1547, and 1339 days, respectively. Survival in oligoastrocytoma and oligodendroglioma tumors present similar, albeit weaker (χ2Mental-Cox=6.06, df=2, P=0.0483, χ2Mental-Cox=4.468, df=2, P=0.1071, respectively), negative correlations to palladin expression.

In embodiments, the TCGA-GBMLGG dataset was then reanalyzed using the new WHO CNS5 classification. Overall, 152 IDH-mutant, and 1p/19q-codeleted oligodendroglioma tumors, 234 IDH-326 mutant astrocytoma tumors, and 186 IDH-wildtype glioblastoma tumors were identified. Significant differences were observed in mean palladin expression between the new classifications (HKruskal-Wallis=287.5, 328 P<0.0001). Glioblastoma tumors express 150% and 70% more palladin than oligodendroglioma, and astrocytoma tumors, respectively (P<0.0001 and P<0.0001, respectively). When the 5-year overall survival of the newly classified tumors were reanalyzed, significant palladin dependent variation within each type was not observed.

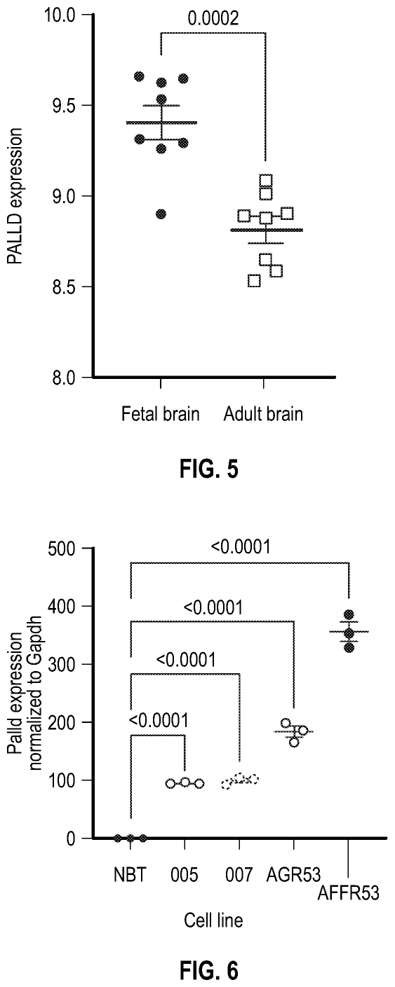

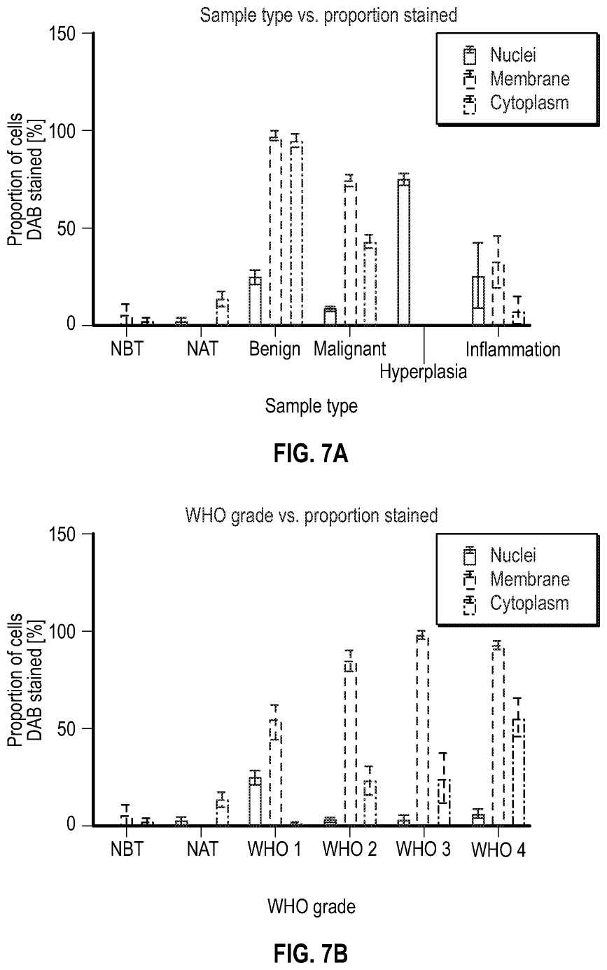

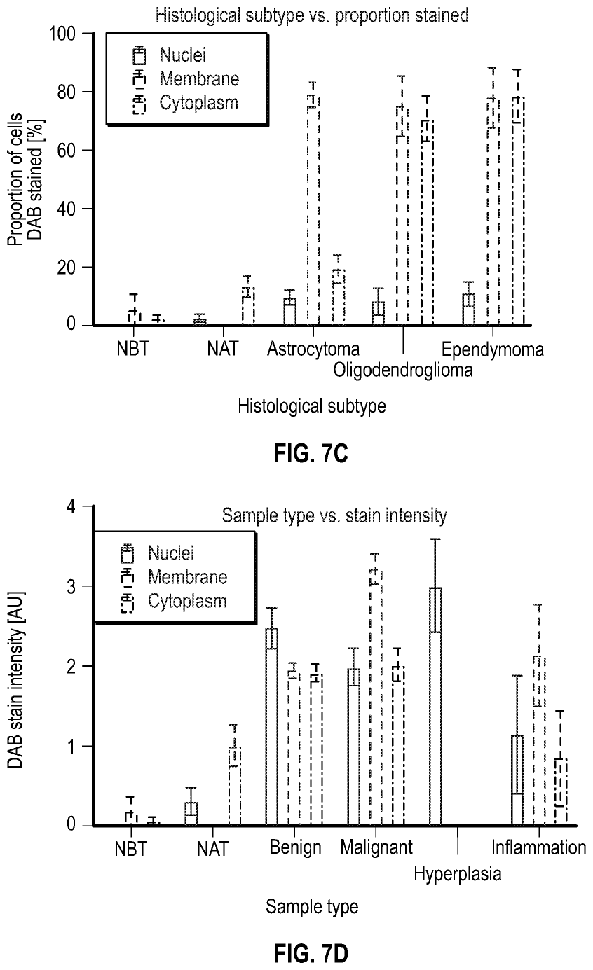

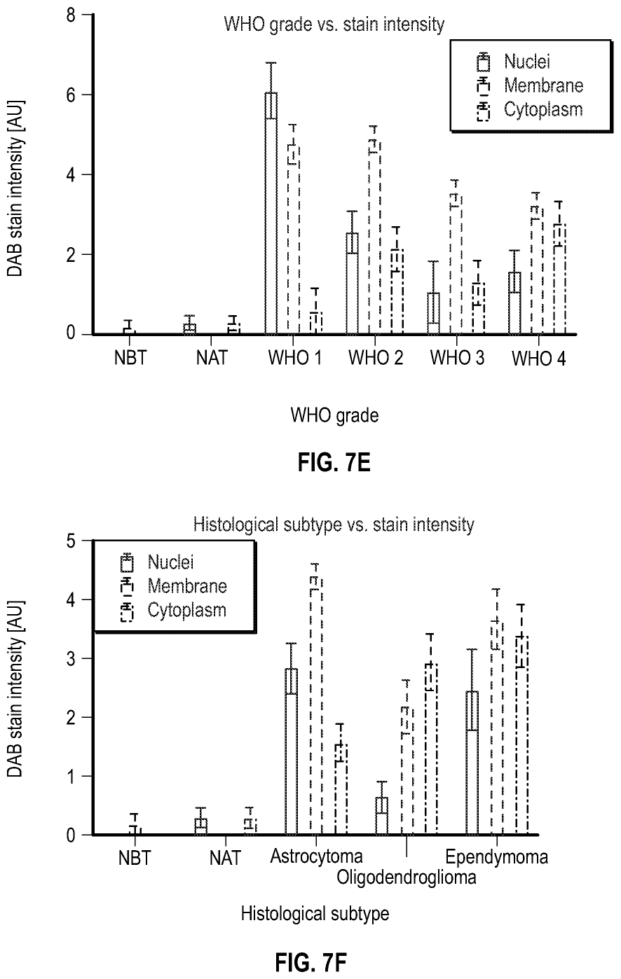

In embodiments, FIG. 5 provides a comparison of palladin expression in fetal brains and adult brains. FIG. 6 shows a qRT-PCR analysis of palladin expression in murine glioblastoma cells and normal brain tissue. FIG. 7A is an analysis of tissue microarray stained with an a-palladin antibody (sample type vs. proportion stained). Staining is shown in respect to sample type (left column), histological grade (middle column), and histological subtype (right column). Proportion (top row) and intensity (bottom row) are shown of stained nuclei (black), membrane (red), and cytoplasm (green). FIG. 7B is an analysis of tissue microarray stained with an a-palladin antibody (WHO grade vs. proportion stained). FIG. 7C is an analysis of tissue microarray stained with an α-palladin antibody (histological subtype vs. proportion stained). FIG. 7D is an analysis of tissue microarray stained with an α-palladin antibody (sample type vs. stain intensity). FIG. 7E is an analysis of tissue microarray stained with an α-palladin antibody (WHO grade vs. stain intensity). FIG. 7F is an analysis of tissue microarray stained with an α-palladin antibody (histological subtype vs. stain intensity).

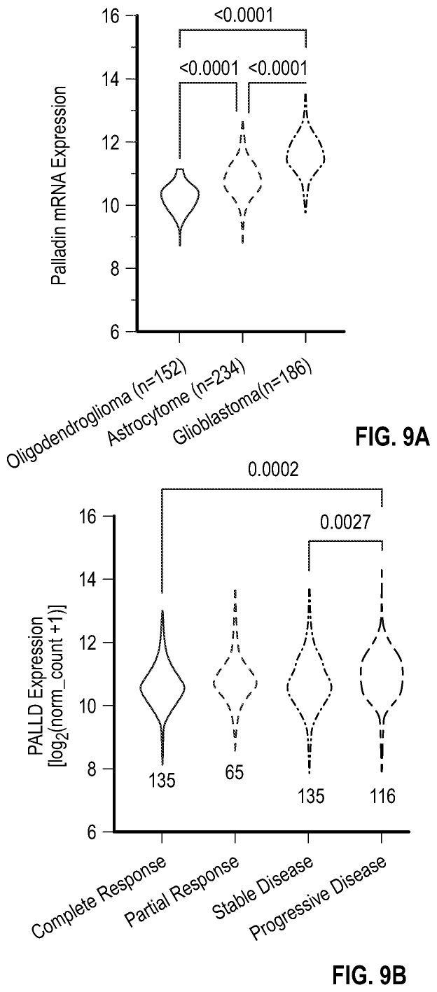

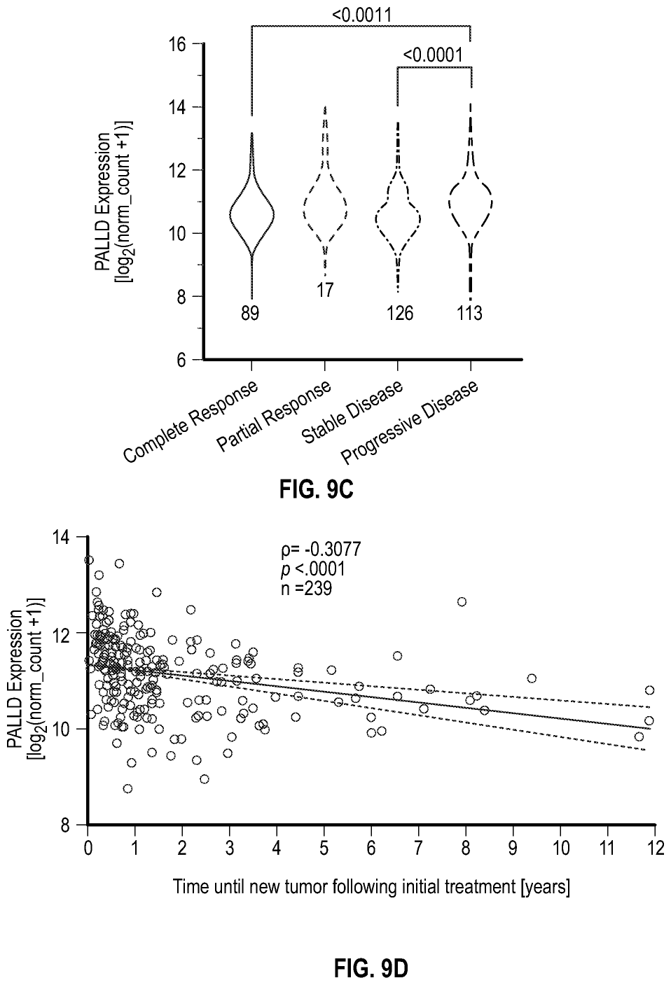

In embodiments, aggressive glioma tumors were shown to be characterized by higher levels of paladin. To determine whether palladin expression correlates with tumor progression or response to treatment, clinical and histopathologic data were analyzed. Palladin overexpression was observed in tumors presenting with progressive disease compared to stable disease and to complete remission following primary treatment (F=6.825, P=0.0002, P=0.0027, and P=0.0002, respectively, FIG. 9B). Similar results were noticed in tumors after follow-up treatment (F=8.692, P<0.0001, P<0.0001, and P=0.0011, respectively, FIG. 9C). Plotting the time to tumor recurrence against palladin expression produces a significant negative correlation (rPearson=−0.3077, P<0.0001, FIG. 9D). This indicates that faster recurring tumors express higher levels of palladin. Further validation of palladin's role in tumor aggression was obtained by analyzing the level of palladin expression in newly classified astrocytoma tumors across different WHO CNS5 grades. A clear increase in palladin transcription was observed as the grade increased, with WHO CNS grade 4 samples expressing 40% more than WHO CNS grade 2 tumors (HKruskal-Wallis=8.304, P=0.0157, PAdj=0.0217).

FIG. 8A shows representative ×20 images of the tissue microarray from FIGS. 7A-7F. FIG. 8B shows representative ×63 images of α-palladin antibody-stained murine tumor and normal brain tissue. FIG. 9A shows an analysis of palladin expression in oligodendroglioma, astrocytoma, and glioblastoma tumors. FIG. 9B shows an analysis of palladin expression in complete response, partial response, stable disease, progressive disease scenarios. FIG. 9C shows a one-way ANOVA with Tukey multiple comparison tests. FIG. 9D is a Pearson correlation test, showing a simple regression line in red with 95% confidence interval, showing time until new tumor following initial treatment.

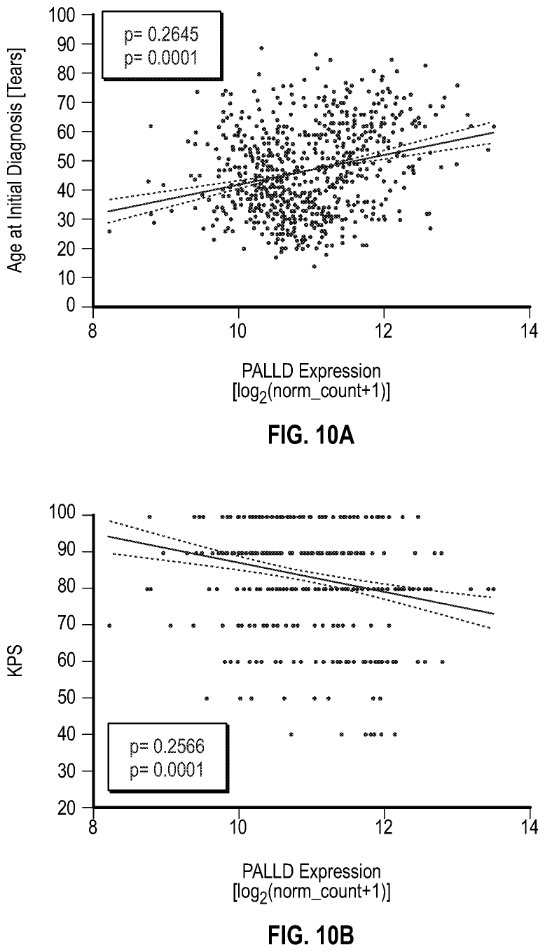

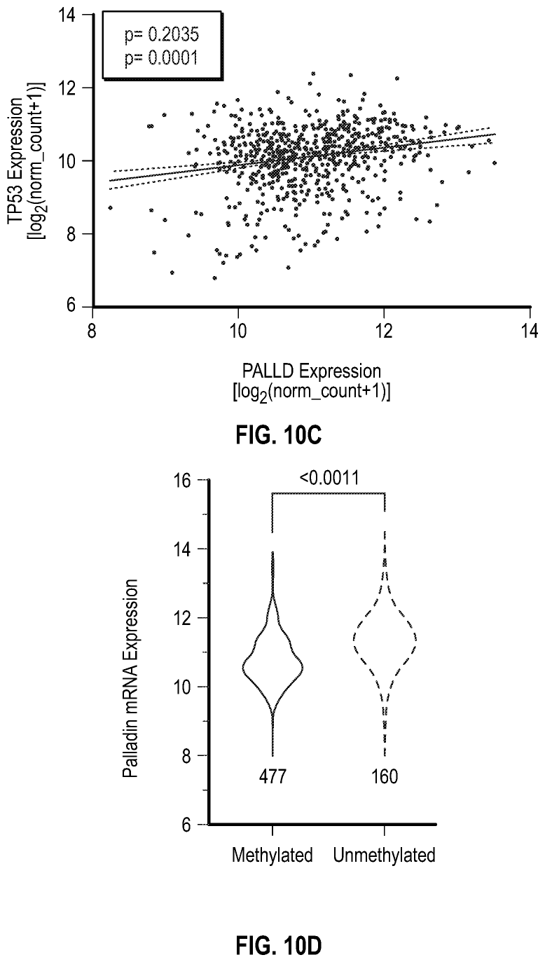

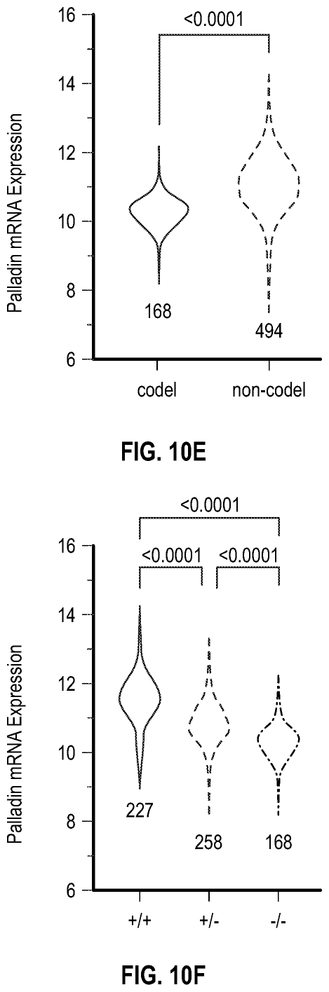

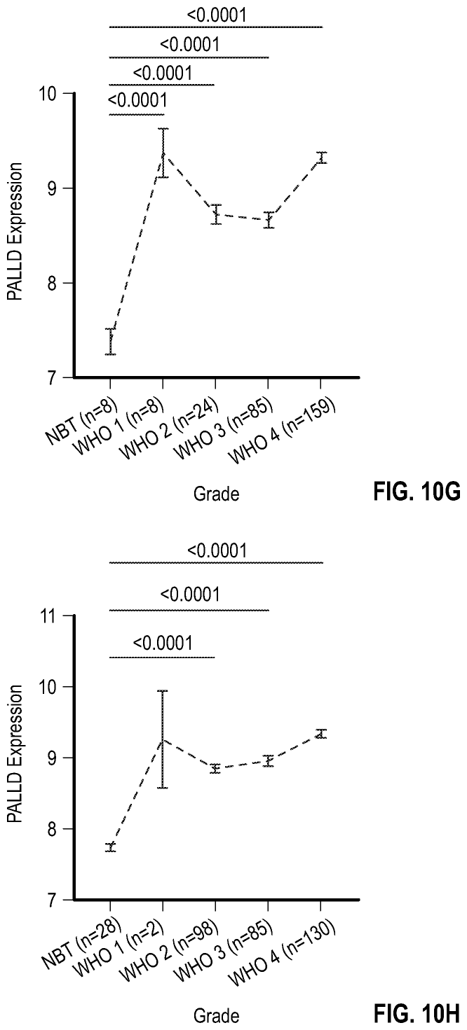

In embodiments, palladin expression was compared to commonly used diagnostic and prognostic markers. Next, the expression of palladin was analyzed and its predictive value in established diagnostic and prognostic markers of glioma was determined. Palladin expression was first plotted against patient age, KPS, and TP53 expression. PALLD expression was significantly positively correlated with age at diagnosis and TP53 levels (rSpearman=0.2645, n=695, P<0.0001 and rSpearman=0.2035, n=702, P<0.0001, respectively, FIGS. 4A-B). Palladin levels were inversely correlated with KPS (rSpearman=−0.2566, n=439, P<0.0001, FIG. 10B).

In some embodiments, a comparison was then made of PALLD expression levels of samples with and without MGMT promoter methylation, and with the presence or absence of a 1p/19q chromosomal codeletion. In embodiments, palladin was significantly overexpressed in tumors with an unmethylated MGMT promoter (UMann-Whitney=21063, P<0.0001, FIG. 10D). Similarly, tumors with 1p/19q codeletion exhibited an almost two-fold increase in palladin expression compared to non-codeletion samples (UMann-Whitney=12362, P<0.0001, FIG. 10E). Furthermore, palladin expression increased as IDH1 alleles were lost (HKruskal-Wallis=291.1, P<0.0001, FIG. 10F), and reached a level doubled that of the baseline when both alleles were lost. Lastly, a multivariate Cox regression model was fit to the overall survival data using the current clinical markers of glioma prognosis available in the TCGA-GBMLGG dataset, with and without palladin expression as a covariate.

FIG. 10A shows the correlation of palladin expression with patient age at diagnosis. FIG. 10B shows the correlation of palladin expression with patient age and Karnofsky performance score (KPS). FIG. 10C shows the correlation of palladin expression with patient age and TP53 expression. FIGS. 10A-C together show that palladin is a diagnostic and prognostic marker of glioma tumors. FIG. 10D shows an analysis of palladin expression with respect to MGMT promoter status. FIG. 10E shows an analysis of palladin expression with respect to chromosome 1p/19q codeletion status. FIG. 10F shows an analysis of palladin expression with respect to IDH1 deletion status. FIG. 10G shows palladin transcription levels in normal brain tissue (NBT) and glioma tumors ranging from grades 1-4. FIG. 10H also shows palladin transcription levels in glioma tumors and NBT ranging from grades 1-4.

In embodiments, a naive model that includes patient age, KPS, TP53 expression, and the presence of IDH1 mutation yielded a concordance score of 0.874 (Table 2). The presence of the IDH1 mutation was the most predictive covariate, with a hazard ratio (HR) of 0.1285 (P=0.000933, 95% CI-0.03814 to 0.4331). A model including palladin mRNA expression was then fitted to the survival data; this produced a 0.92 concordance score statistic (Table 3). PALLD expression (P=0.00365, 95% CI=1.518 to 8.548) and age at diagnosis (P=0.01083, 95% CI=1.016 to 1.134) proved the most influential markers for patient survival, with HRs of 3.6023 and 1.0736, respectively. Using a likelihood ratio test to compare the two models, the model incorporating palladin proved superior (χ2likelihood ratio test=9.7542, df=1, P=0.001789, Table 4). A third model was fitted with only the significant covariates but this model performed worse than the other two, as the concordance score was only 0.852.

In embodiments, to examine whether palladin can be used in the diagnosis or risk stratification of glioma tumors, all available datasets that included data about palladin expression, histological classification, and grade in non-tumor, LGG, and GBM samples were analyzed. Overall, 2638 samples were analyzed from one pediatric and four adult datasets. Of the five datasets, only three included samples of WHO CNSA grade 1 tumors, which were crucial for this type of analysis. In all three datasets, PALLD expression was significantly greater in tumor than non-tumor samples. In two of the datasets, PALLD expression was significantly greater in WHO CNSA grade 1 tumors than in normal brain tissue (NBT).

TABLE 2
Results of the multivariate Cox regression model - not including palladin.

| | | Standard | | | Lower | Upper |
|---|---|---|---|---|---|---|
| Covariates | Coefficient | Error | P | HR | CI_{95%} | CI_{95%} |
| TP53 | 0.42027 | 0.50473 | 0.405035 | 1.5224 | 0.5661 | 4.0939 |
| Age at diagnosis | 0.4777 | 0.0245 | 0.051199 | 1.0489 | 0.99975 | 1.1005 |
| KPS score | −0.0442 | 0.03717 | 0.234326 | 0.9568 | 0.88954 | 1.0291 |
| IDH1 mutation | −2.05162 | 0.61982 | 0.000933 | 0.1285 | 0.03814 | 0.4331 |
| (0: no, 1: yes) | | | | | | |

HR = Hazard Ratio,
CI = Confidence Interval,
KPS = Karnofsky Performance Score

TABLE 3
Results of the multivariate Cox regression model - including palladin.

| | | | | | Lower | Upper |
|---|---|---|---|---|---|---|
| Covariates | Coefficient | Standard Error | P | HR | CI_{95%} | CI_{95%} |
| PALLD | 1.281586 | 0.440867 | 0.00365 | 3.6023 | 1.51817 | 8.548 |
| TP53 | 0.846903 | 0.600697 | 0.15858 | 2.3324 | 0.71861 | 7.57 |
| Age at diagnosis | 0.070992 | 0.027862 | 0.01083 | 1.0736 | 1.01652 | 1.134 |
| KPS score ^{a} | −0.008419 | 0.040141 | 0.83388 | 0.9916 | 0.91659 | 1.073 |
| IDH1 mutation (0: no, 1: yes) | −1.319920 | 0.680868 | 0.05255 | 0.2672 | 0.07034 | 1.015 |

HR = Hazard Ratio, CI = Confidence Interval

TABLE 4
Results of the multivariate Cox regression
model - including palladin.

| #DF | Log Likelihood | DF | χ² | P |
|---|---|---|---|---|
| 4 | −39.743 | | | |
| 5 | −34.866 | 1 | 9.7542 | 0.001789 |

DF = Degrees of Freedom

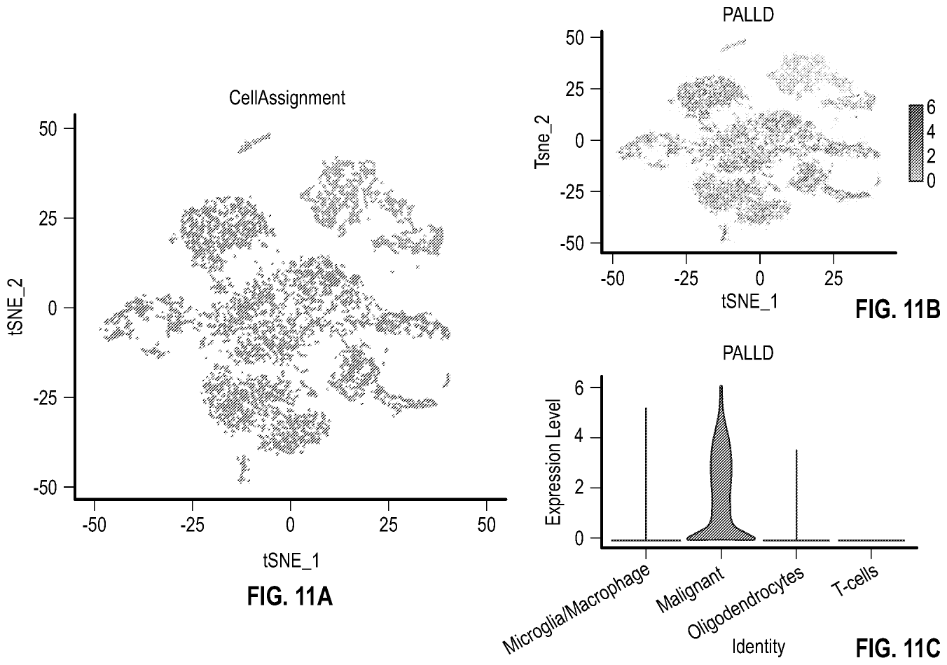

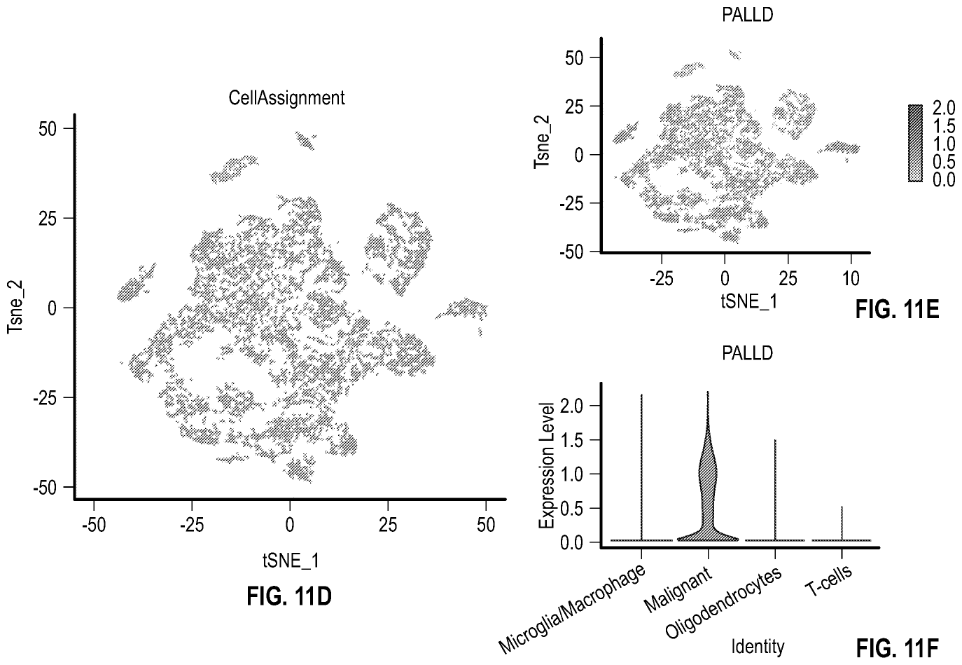

In embodiments, palladin is overexpressed principally in malignant cells and not in other glioma-related cell types. To examine the origin of palladin expression in glioma tumors, single-cell RNA sequencing data of astrocytoma and GBM tumors were obtained and analyzed. According to the disclosure, 6225 cells were analyzed. The cells originated from 10 IDH-mutant astrocytoma tumors designated as one of four cell types: malignant cells, microglia/macrophages, oligodendrocytes, or T cells. Palladin expression was detected in 71.6% of the malignant cells, 11.8% of microglia/macrophages, and 6.1% of oligodendrocytes (FIGS. 11A-C). In these respective samples, PALLD expression was 1.65, 0.18, and 0.09 times the mean level of the entire cohort. No palladin mRNA was identified in the T cell population. Next, 7930 cells with identical cell classes in 28 GBM tumors were analyzed. Palladin was observed in 62.2% of the malignant cells, 24.7% of microglia/macrophages, 4.6% of oligodendrocytes, and 1.1% of T cells (FIGS. 5E-H). In these respective samples, PALLD levels were 1.36, 0.56, 0.07, and 0.01 times the mean PALLD level of the entire cohort.

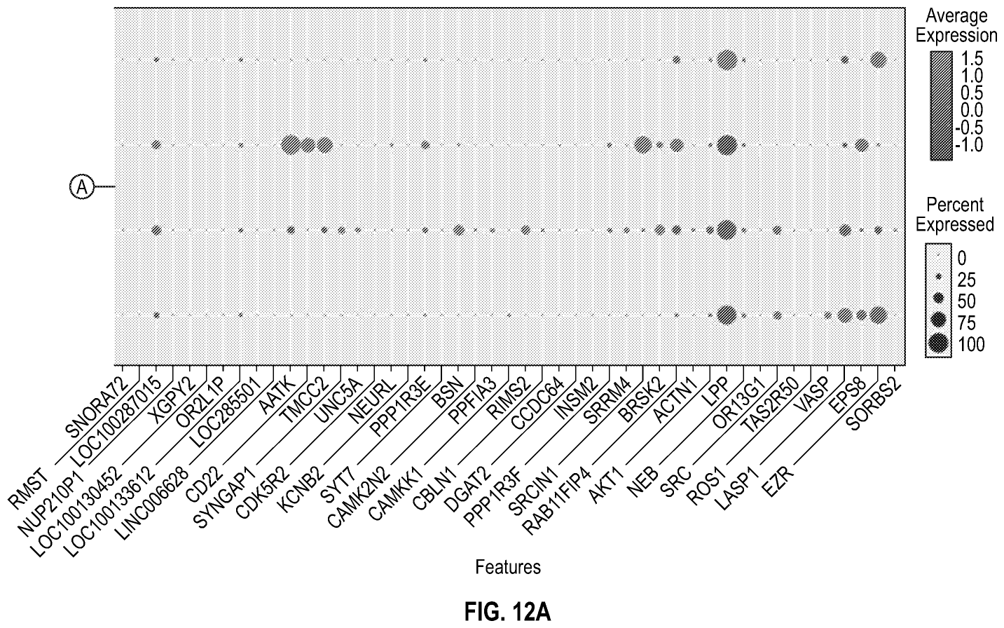

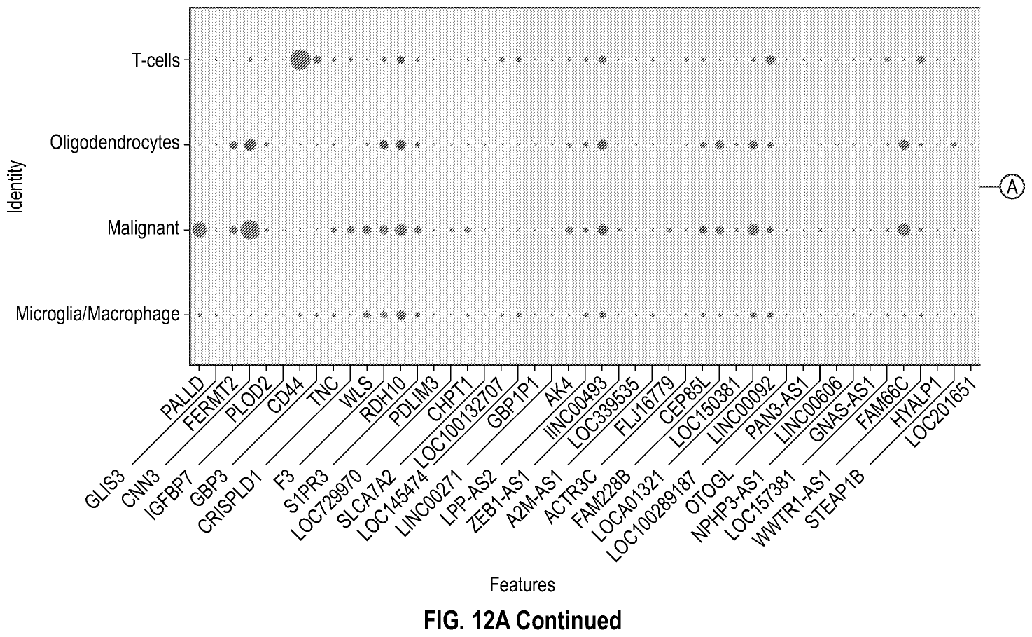

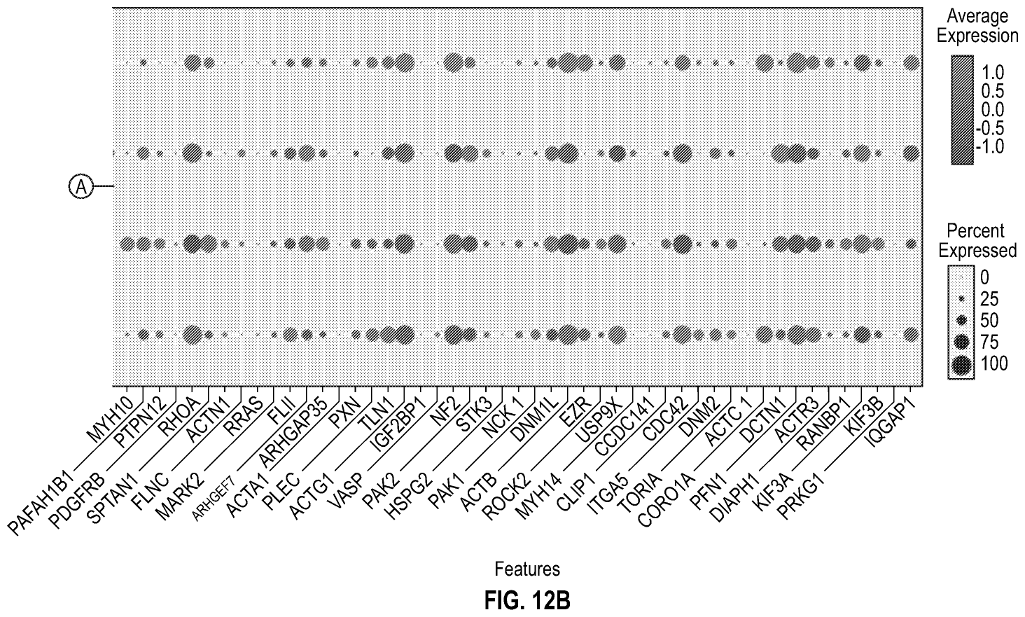

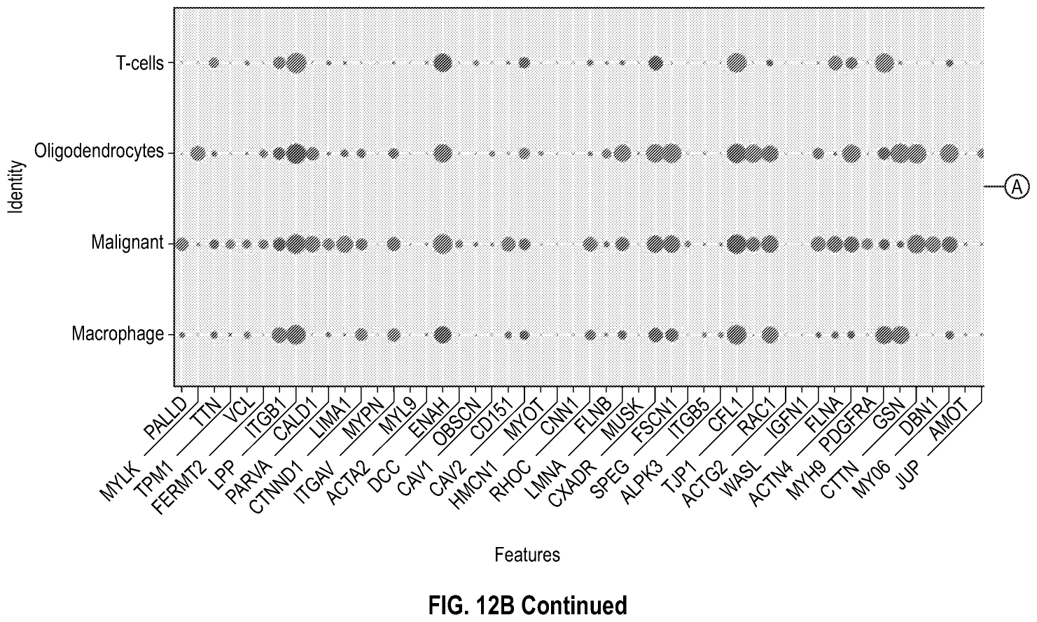

In some embodiments, palladin was compared to other similar genes to determine whether its pattern of expression is specifically associated with gliomas. A list of 100 genes that are similar to palladin was formulated using Gene Card Suite's Genes Like Me algorithm. The similarity score was based on the relatedness between two genes, of their domains, sequence paralogy, expression patterns, modulating compounds, super pathways, phenotypes, gene ontology, and associated disorders. Of the 100, data were available for 98 genes in the two studies used in our single cell analysis. The genes were filtered by whether their mean expression and proportion of expressing cells in the malignant cell population were equal to or higher than in PALLD (FIGS. 12A-12B).

FIG. 11A shows t-distributed stochastic neighbor embedding (t-SNE) scatter plots of single cell RNA sequencing (scRNAseq) astrocytoma data shaded according to cell type. FIG. 11B shows t-distributed stochastic neighbor embedding (t-SNE) scatter plots of single cell RNA sequencing (scRNAseq) astrocytoma data shaded according to origin of data. FIG. 11C shows t-distributed stochastic neighbor embedding (t-SNE) scatter plots of single cell RNA sequencing (scRNAseq) astrocytoma data shaded according to palladin expression. FIG. 11D show t-SNE scatter plots of scRNAseq glioblastoma multiforme (GBM) data colored according to origin of data, cell type, and palladin expression. FIG. 11E show t-SNE scatter plots of scRNAseq glioblastoma multiforme (GBM) data colored according to palladin expression. FIG. 11F show expression level data according to cell type.

In embodiments, an evaluation was conducted into whether the pattern of expression of each of the filtered genes was unique to the malignant cell population (e.g., not found in the other cell types). Five of the filtered genes passed this criterion in both studies: LIMA1, WASL, DBN1, MYH10, and SPTAN1. These genes all mimic palladin's single-cell expression pattern. The impact of these gene expression levels on the overall survival of adults with glioma in the TCGA-GBMLGG cohort was analyzed. In embodiments, LIMA1 and WASL showed no significant effect on overall survival, while DBN1, MYH10, and SPTAN1 all increased survival, with a concordant increase in their expression.

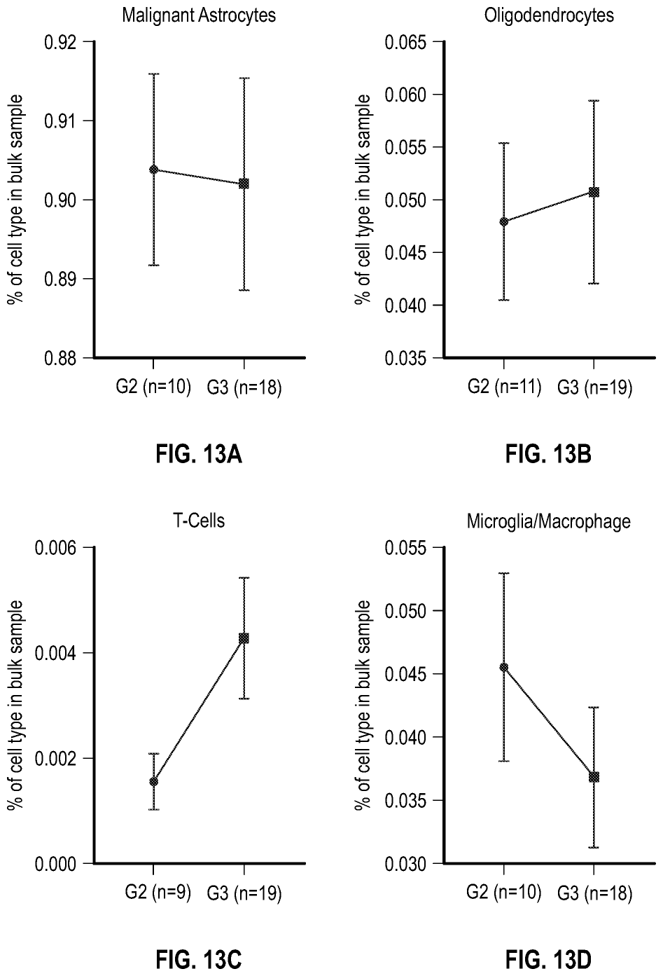

In embodiments, in-silico flow cytometry was used to investigate if the increase in palladin expression might originate from changes in various cell population proportions within the tumor as it progresses. In embodiments, analysis of cell proportions in grades II and III IDH1-mutated astrocytoma tumors (n=32) using single cell and bulk RNAseq data revealed no significant changes in the proportions of malignant astrocytes, macrophages, oligodendrocytes, and T-cells within a tumor, between grades II and III (FIGS. 13A-13D).

FIGS. 11A-11D & 12A-12B illustrate that palladin is uniquely expressed in the malignant cell population of glioma tumors. FIGS. 11A-B show t-distributed stochastic neighbor embedding (t-SNE) scatter plots of single cell RNA sequencing (scRNAseq) astrocytoma data colored according to cell type, origin of data, and palladin expression, respectively. FIG. 11C provides quantification of palladin expression in different cell types in astrocytoma scRNAseq data. FIGS. 11D-11E show t-SNE scatter plots of scRNAseq glioblastoma multiforme (GBM) data colored according to origin of data, cell type, and palladin expression. FIG. 11F provides quantification of palladin expression in different cell types in GBM scRNAseq data. FIGS. 12A & 12B provides expression quantification of palladin in 100 similar genes in different cell types in astrocytoma and GBM scRNAseq data, respectively. (FIGS. 11A, 11C, D11, 11F) Malignant cells are colored in green, oligodendrocytes in teal, T-cells in purple and microglia/macrophages in red.

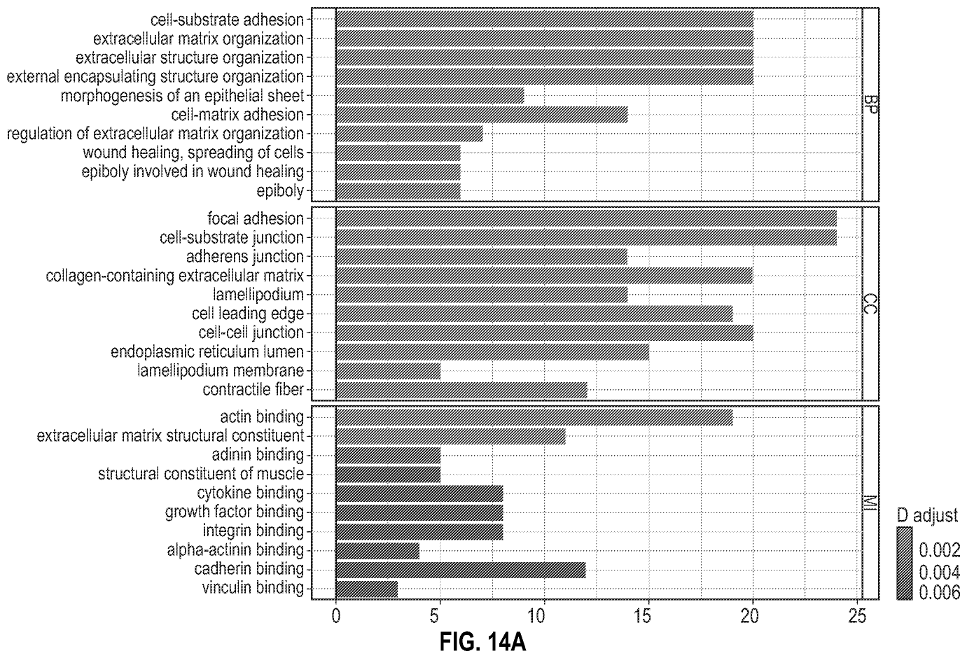

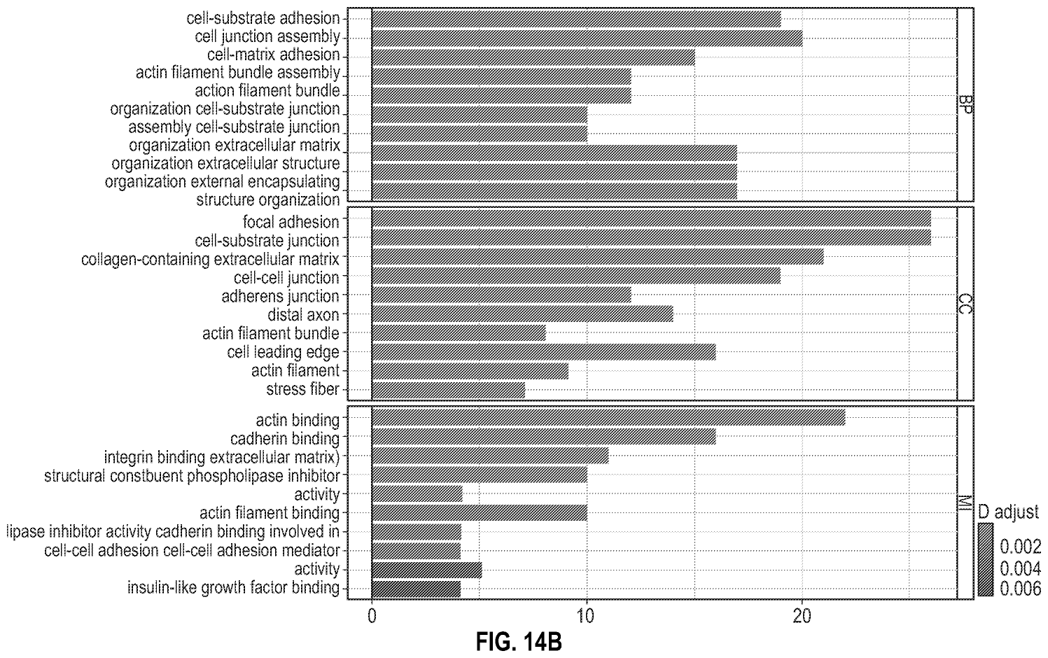

In embodiments, palladin is related to a transcriptional program involved in cellular motility and the extracellular matrix. To gain insight into palladin's role in transcriptional regulation, gene co-expression with PALLD was analyzed using the SEEK [Human] server. A total of 40 datasets were used for the co-expression analysis in astrocytoma tumors and 100 datasets were used for GBM. In embodiments, in the astrocytoma datasets, 193 significantly (α=0.01) co-expressed genes were identified, while 155 genes were found in GBM datasets. In embodiments, overrepresentation analysis of gene ontology terms from a search of the significant genes yielded terms related to the extracellular matrix, actin cytoskeleton, and cellular motility (FIGS. 14A-14B).

FIG. 12A shows expression quantification of 100 genes similar to Palladin in a number of cell types in astrocytoma and GBM scRNAseq data. FIG. 12B also shows expression quantification in various genes similar to Palladin in a number of cell types in astrocytoma and GBM scRNAseq data. FIG. 13A shows in-silico flow cytometry of grades 2-3 IDH1-mutant astrocytoma tumors for malignant astrocytes. FIG. 13B shows in-silico flow cytometry of grades 2-3 IDH1-mutant astrocytoma tumors for oligodendrocytes. FIG. 13C shows in-silico flow cytometry of grades 2-3 IDH1-mutant astrocytoma tumors for T-cells. FIG. 13D shows in-silico flow cytometry of grades 2-3 IDH1-mutant astrocytoma tumors for microglia/macrophages. FIG. 14A shows a gene Ontology enrichment analysis of genes significantly co-expressed with palladin in astrocytoma datasets. Many motility-related terms are observed. FIG. 14B shows a gene Ontology enrichment analysis of genes significantly co-expressed with palladin in glioblastoma multiforme datasets. Many motility-related terms are observed.

In embodiments, palladin is disclosed to be a driver of breast cancer metastasis and appears to play a promoting role in other cancers as well. In embodiments, palladin is significantly upregulated in bile duct, brain, breast, liver, lung, pancreas, stomach, and thyroid cancers; and it is downregulated in tumors of the bladder, cervix, colon, endometrium, esophagus, ovary, prostate, rectum, skin, testis, and uterus. In embodiments, palladin is overexpressed in pancreatic and breast cancers. In embodiments, cancers of muscular organs or organs rich in muscle tissue (e.g. the endometrium and colon, respectively) exhibited decreased palladin expression. In embodiments, paladin regulates the differentiation and maturation process of healthy muscle cells. In embodiments, testicular cancers also exhibit decreased palladin levels relative to healthy tissues in which palladin has a functional role. This can be explained by changing transcription patterns or relative proportions of palladin-expressing cells within the tumor as it progresses.

In further embodiments, overall survival analysis was performed to narrow the search to tumors that might be affected by palladin expression in a clinically relevant manner. In embodiments, palladin expression is tightly correlated only with the overall survival of individuals with glioma. This result is surprising, as associations of palladin with the survival of individuals with breast and pancreatic cancers was also expected as palladin can contribute to aggressive behavior by promoting cell invasion in these cancers. In embodiments, palladin is essential in the morphology of reactive astrocytes, which contribute to the progression of glioma tumors. Analysis of gene expression at the isoform level revealed that the 90 kDa palladin transcript 4 was the most abundant in healthy brain tissue and that its expression increased concordantly with the grade of the tumors analyzed. These results suggest that palladin's role in glioma tumors is related to its F-actin bundling capabilities mediated by immunoglobulin (Ig) tandem domains 3 and 4, which are present in this isoform. In embodiments, palladin also acts indirectly in gliomas via interactions with its binding partners. Additional experiments that knockdown palladin in-vitro can be useful in establishing specific palladin characteristics.

According to the present disclosure, the search for somatic mutations in palladin's sequence and the absence of any in the vast majority of glioma tumors suggests that non-mutated palladin is crucial for cellular function and tumor development. This is consistent with palladin's importance in maintaining proper cellular shape, motility and invasiveness, cell division, and embryonic development. As the WHO CNS5 classification considers pediatric and adult gliomas as distinct pathological types, the patterns of palladin expression in pediatric glioma datasets were also analyzed. Results from those datasets did not confirm the results of adult gliomas. In embodiments, palladin expression in fetal brains was significantly higher than in adult brains. While still requiring further validation, as palladin levels are increased in tumors, this observation can explain the lack of a significant difference between tumor and healthy pediatric tissue.

In embodiments, from images of healthy and tumor mouse tissues, the findings of increased palladin mRNA levels in tumors compared to healthy brain tissue were validated at the protein level, and a correlation was established between this increase and WHO CNS5 grade. Fluorescent imaging showed palladin's localization to the site of glioblastoma cell injection in healthy mouse brain tissue. This finding was supported by qRT-PCR, which revealed palladin overexpression in glioblastoma cell cultures compared to NBT. Palladin staining patterns appeared to include the neutrophil and membrane of tumor cells, as well as the cell edges. This raises the possibility of using palladin to delineate glioma tumors, similar to the use of 8-aminolevulinic acid in fluorescence-guided neurosurgery.

In other embodiments, human TMA of CNS pathologies were stained. In embodiments, while palladin is present in the nuclei and cytoplasm of cells in the CNS, its presence in the membrane is indicative of a pathology. Palladin's proportion and intensity of membranal staining were greater in benign and malignant tumors than in healthy, hyperplastic, and inflamed tissues. In gliomas, the level of palladin detected in the membrane increased in correlation with the WHO grade. This observation was not restricted to a specific tumor type. The results of the present disclosure also indicate that glioblastoma tumors are the most prone to palladin overexpression. This raises the that palladin expression in glioma tumors can be used for diagnostic purposes.

In some embodiments, survival of patients with astrocytoma decreases as palladin levels increase, in a dose response manner. Classifying adult glioma tumors according to the updated WHO CNS5 guidelines made differences in five-year survival independent on palladin expression within each tumor group (but not across the groups). In embodiments, palladin is a tool for molecular diagnosis and differentiation of adult type gliomas. In embodiments, paladin expression predicts the survival of patients. In other embodiments, palladin expression accurately predicts the tumor type with deadlier tumors expressing more palladin.

As described above, the present disclosure demonstrates a link between palladin expression levels, poor treatment response, and earlier recurrence. To assess the for clinical applications of palladin, the rapidity of the increase in palladin level within a tumor was examined. In embodiments, palladin expression appears to rise immediately when the tissue is transformed and the tumor is classified as grade 1. In embodiments, palladin expression serves as a diagnostic marker for astrocytomas.

In embodiments, palladin serves as a prognostic marker. In embodiments, the present disclosure demonstrates a correlation of palladin to common prognostic features such as patient age at diagnosis and TP53 expression, and an inverse correlation with KPS. Furthermore, Cox multivariate regression was used to analyze palladin's association with mortality. The results indicate that the level of palladin expression is a stronger predictor than the currently used prognostic markers, of the overall survival of individuals with gliomas.

In further embodiments, scRNAseq data from astrocytoma and GBM tumors was analyzed. In embodiments, palladin expression originates from the malignant astrocytes and GBM tumor cells. To validate the findings of the present disclosure, the expression of 100 genes genetically, phenotypically, structurally, and transcriptionally similar to palladin were investigated. These results reinforce the uniqueness and clinical relevance of palladin's transcription patterns in glioma tumors.

In embodiments, the disclosure also relates to the treatment of glioma tumors through downregulation of palladin or inhibition of its binding to actin cytoskeleton. There are several approaches to inhibit palladin's binding to actin including direct inhibition wherein a small molecule designed and used to block the Palladin: Actin binding site, and indirect inhibition wherein a small molecule designed and used to block the Palladin: Palladin binding site, which should have a similar effect as direct inhibition.

In embodiments, Glioblastoma are highly invasive while being less prone to distant metastasis. Invasion is a complex process involving the loss of cellular adhesion, epithelial-to-mesenchymal transition (EMT), increase in cell motility, and degradation and reorganization of the surrounding extracellular matrix (ECM). In embodiments, hypoxia and inflammation related signaling pathways regulate this invasion. In embodiments, inflammation and subsequent myeloid cell recruitment increase transforming growth factor beta (TGF-b) and platelet derived growth factor (PDGF) expression and signaling. Both of the above lead to EMT and rely on palladin downstream. Palladin is tied to the hypoxia related PI3K/AKT/mTOR pathways, and hypoxia can trigger invasion in glioblastoma. Kinases, AKT1 and 2, can modulate palladin f-actin binding activity and expression, respectively. The SRC proto-oncogene non-receptor tyrosine kinase (SRC) is another pathway capable of remodeling the cytoskeleton in response to hypoxic stimuli. Further, SRC mediated, PDGF induced membrane ruffling and lamellipodia formation required both palladin and SPIN90. According to the present disclosure, downregulation of palladin or inhibition of its binding to the actin cytoskeleton might curb the aggressive phenotype of glioma tumors. Nevertheless, additional experimental work is needed to determine the extent of clinical value.

In some embodiments, a method of treating brain tumors in a subject is disclosed. In embodiments, detecting or diagnosing glioma in the subject includes: a) measuring PALLD mRNA levels in a plasma sample of the subject; correlating the PALLD mRNA levels to PALLD mRNA plasma levels corresponding to a likelihood of having a specific type of glioma; making a diagnosis based on the PALLD mRNA levels of the plasma sample as compared to the expected PALLD mRNA plasma levels corresponding to a likelihood of having a specific type of glioma; and b) treating subject diagnosed with specific type of glioma or having the likelihood of developing specific type of glioma with at least one therapeutic agent for treating glioma. In other embodiments, disclosed is a method of treating brain tumors wherein the at least one therapeutic agent (e.g., miR-96 and miR-182, or the like) causes downregulation of palladin within targeted tissue of a subject. In yet another embodiment, the at least one therapeutic agent causing downregulation of palladin comprises miR-96 and miR-182, wherein the miR-96 and miR-182 are bound to gold nanoparticles and/or and are further embedded in a hydrogel.

In another embodiment, the therapeutic agent includes at least one miRNA molecule selected from the group consisting of miR-96 and miR-182, or at least one vector expressing or encoding the same, for use in reducing or preventing the specific type of glioma. In other embodiments, the therapeutic agent is formulated for systemic administration, local administration, intra-tumor administration, enteral administration, oral administration, sublingual and buccal administration, rectal administration, intravenous administration, intramuscular administration, and/or subcutaneous administration.

In some embodiments, the glioma comprises a cancer associated with abnormal palladin expression and/or activity. In other embodiments, the glioma is an ependymoma, the glioma is selected from the group consisting of astrocytoma or oligodendroglioma, or the glioma comprises a glioblastoma (or another high-grade glioma originating in astrocytes). In other embodiments, the subject is at risk of developing metastasis and the administering is carried out prior to metastasis formation. In yet another embodiment, the subject has already developed metastases and the administering is carried out after metastasis formation.

In other embodiments, a method of treating brain tumors is disclosed wherein a therapeutic agent inhibits the binding of palladin to actin cytoskeleton (e.g., actin binding proteins, Jasplakinolide, Latrunculin B, and the like). In embodiments, over 150 actin-binding proteins (ABPs) are known that may influence localization, polymerization dynamics, crosslinking, and organization of actin. These ABPs have various means of regulating actin, which include the ability to sequester monomeric actin (G-actin), nucleate filament formation, sever filamentous actin (F-actin), generate branched arrays of actin, and cap actin filaments.

In embodiments, glioblastoma tumors express approximately 150% and 70% more palladin than oligodendroglioma, and astrocytoma tumors, respectively (P<0.0001 and P<0.0001). In other embodiments, glioblastoma tumors express approximately (100%, 125%, 150%, 175%, or 200%) and (50%, 70%, 90%, 100%, 140%) more palladin than oligodendroglioma, and astrocytoma tumors, respectively (P<0.0001 and P<0.0001).

In still other embodiments, a method of detecting or diagnosing glioma tumor type in a subject is disclosed. Said method includes the steps of measuring PALLD mRNA levels in a plasma sample of the subject; correlating the PALLD mRNA levels to PALLD mRNA plasma levels corresponding to a likelihood of having a specific type of glioma; and making a diagnosis based on the PALLD mRNA levels of the plasma sample as compared to the expected PALLD mRNA plasma levels corresponding to a likelihood of having a specific type of glioma. In other embodiments, said method of detecting or diagnosing glioma comprises an adult-type diffuse glioma. In another embodiment, a method is disclosed delineating brain tumor tissue from surrounding brain, the method comprising injecting a palladin-associating fluorophore or prodrug comprising a palladin-associating fluorophore into a subject for fluorescence-guided neurosurgery.

As described above, in embodiments, a method for detecting or diagnosing glioma in a subject is disclosed. The method includes measuring PALLD mRNA levels in a plasma sample of the subject. The PALLD mRNA levels can be measured using a variety of techniques, such as quantitative PCR (qPCR), microarray analysis, or RNA sequencing. In embodiments, the method includes correlating the PALLD mRNA levels to PALLD mRNA plasma levels corresponding to a likelihood of having a specific type of glioma. This correlation can be established using a variety of statistical methods, such as logistic regression or receiver operating characteristic (ROC) analysis. The method includes making a diagnosis based on the PALLD mRNA levels of the plasma sample as compared to the expected PALLD mRNA plasma levels corresponding to a likelihood of having a specific type of glioma. In embodiments, the method may also include a diagnosis made by a physician or other healthcare professional based on the results of the PALLD mRNA test and other clinical factors.

In other embodiments, agents can be siRNAs, antibodies, small inhibitory molecules, aptamers and the like that downregulate palladin or inhibit of its binding to the actin cytoskeleton. For example, the therapeutic agent can be an inhibitor such as a siRNA or an antibody to downregulate palladin or inhibit of its binding to the actin cytoskeleton. Examples of therapeutic agents include, but are not limited to siRNA, antibodies, ligands, recombinant proteins, peptide mimetics, and soluble receptor fragments. One specific example of a therapeutic agent is a siRNA. Methods of making siRNA that can be used clinically are known in the art. In a particular example, siRNA hybridize to molecules that regulate palladian, directly to palladian itself, or in a manner that inhibits the binding of palladian to the actin cytoskeleton.

In the context of nucleic acid therapeutic agents, a variety of catalytic nucleic acid-assisting reagents, DNAzymes, modified oligonucleotides, and nonnative backbones are also contemplated to down regulate palladian or inhibit its binding to the actin cytoskeleton. In embodiments, said therapeutic agents include various RNAs (such as siRNAs), DNAs (such as ssDNAs), peptide nucleic acids (PNAs), locked nucleic acids (LNAs), and other natural or unnatural polymers are contemplated. For example, polyacrylate analogues of nucleic acids may be used, in addition to nucleobase-containing polymers with a polyester, polyvinyl, or polyamide backbone. Nonnative backbones may include bifacial peptide nucleic acids displaying melamine, for example. Notably, polyacrylate backbones displaying melamine can triplex hybridize efficiently with native bases and nucleic acids, bridging various native and artificial architectures. Additional nucleic acid modifications may be made to the nucleic acid therapeutic agents described herein, including modified riboses. In other embodiments, standard modified oligonucleotides or nucleic acids modifications may be used such as cross-linking, methylation, phosphorothioate incorporation, encapsulation in lipid nanoparticles, and the like. In another embodiment, in vitro selection may be used to obtain optimized nucleic acid based therapeutic molecules such as optimized siRNAs or trans-cleaving ribozymes (e.g., hammerhead ribozyme).

In some embodiments where a therapeutic agent is a nucleic acid molecule (such as an siRNA, shRNA, antisense oligonucleotide, ribozyme or other inhibitory nucleic acid specific for a gene that is upregulated in chemoresistant gastric cancer), administration of the nucleic acid may be achieved in a variety of ways. All forms of nucleic acid delivery are contemplated by this disclosure, including, without limitation, synthetic oligos, naked DNA, naked RNA (such as capped RNA), and plasmid or viral vectors (which may or may not be integrated into a target cell genome). For example, an expressible nucleic acid can be administered by use of a viral vector (see U.S. Pat. No. 4,980,286), or by direct injection, or by use of microparticle bombardment (for example, a gene gun; Biolistic, Dupont), or coating with lipids or cell-surface receptors or transfecting agents, or by administering it in linkage to a homeobox-like peptide which is known to enter the nucleus (see e.g., Joliot et al., Proc. Natl. Acad. Sci., 88:1864-1868, 1991). Alternatively, the expressible nucleic acid can be introduced into a host cell (such as a stem cell, e.g., a stem cell capable of neural differentiation) for expression of a polypeptide therapeutic in the host cell. In some examples, transfected/transformed host cells can be transplanted into a subject. In some instances, a nucleic acid molecule can be incorporated within host cell DNA, for example, by homologous or non-homologous recombination, for stably expressing a therapeutic.

Expression vectors are commonly available that provide, for instance, constitutive, regulated, or cell/tissue-specific expression of a transcribable nucleic acid (e.g., a nucleic acid encoding a chemotherapy sensitivity-related molecule polypeptide) included in the expression vector. All these vectors achieve the basic goal of delivering into the target cell a heterologous nucleic acid sequence and control elements needed for transcription. Regulated expression vectors include control elements that permit expression of an operably linked nucleic acid only when a corresponding regulator molecule (such as tetracycline or steroid hormones) is present. Exemplary regulated vectors include pMAM-neo (Clontech) or pMSG (Pharmacia), which use the steroid-regulated MMTV-LTR promoter, or pBPV (Pharmacia), which includes a metallothionein-responsive promoter. Numerous cell/tissue-specific expression vectors are also available for expression of heterologous nucleic acids in any of a variety of tissues or cell types.

Viral vectors, which are derived from various viral genomes, are similarly numerous and commercially available. Exemplary viral vectors are derived from retroviruses (such as lentivirus), adenovirus, herpes simplex virus (HSV; Margolskee et al., Mol. Cell. Biol. 8:2837-2847, 1988), adeno-associated virus (McLaughlin et al., J. Viral. 62:1963-1973, 1988), polio virus and vaccinia virus (Moss et al., Annu. Rev. Immunol. 5:305-324, 1987). Representative retroviral vectors are derived from lentiviruses, Moloney murine leukemia virus (MoMuLV), Harvey murine sarcoma virus (HaMuSV), murine mammary tumor virus (MuMTV), and Rous Sarcoma Virus (RSV). Multiple teachings concerning viral vectors are available, e.g., Anderson, Science, 226:401-409, 1984; Hughes, Curr. Comm. Mol. Biol., 71:1-12, 1988; Friedman, Science, 244:1275-1281, 1989 and Anderson, Science, 256:608-613, 1992. Some viral vectors are replication-deficient and/or non-infective. Non-limiting representative neurotrophic viral vectors include herpes simplex viral vectors (see, e.g., U.S. Pat. No. 5,673,344) and adenoviral vectors (see, e.g., Barkats et al., Prog. Neurobiol., 55:333-341, 1998), or AAV or lentiviral vectors pseudotyped with rabies-G glycoprotein (Mazarakis et al., Human Mol. Genet., 10:2109-2121, 2001; Azzouz, etal., J. Neurosci., 22:10302-10312, 2002; Azzouz, et al., Nature, 429:413-417, 2004).

Other methods of delivery are also contemplated. For instance, lipidic and liposome-mediated gene delivery has recently been used successfully for transfection with various genes (for reviews, see Templeton and Lasic, Mol. Biotechnol., 11:175 180, 1999; Lee and Huang, Crit. Rev. Ther. Drug Carrier Syst., 14:173-206, 1997; and Cooper, Semin. Oneal., 23:172-187, 1996). For instance, cationic liposomes have been analyzed for their ability to transfect monocytic leukemia cells, and shown to be a viable alternative to using viral vectors (de Lima et al., Mol. Membr. Biol., 16:103-109, 1999). Such cationic liposomes can also be targeted to specific cells through the inclusion of, for instance, monoclonal antibodies or other appropriate targeting ligands (Kao et al., Cancer Gene Ther., 3:250-256, 1996).

In some embodiments, therapeutic agents comprising peptides (e.g., an antibody or fragment thereof) may be delivered by administering to the subject a nucleic acid encoding the peptide. In other embodiments, peptide therapeutic agents may be isolated from various sources and administered directly to the subject. For example, a peptide may be isolated from a naturally occurring source. Alternatively, a nucleic acid encoding the peptide may be expressed in vitro, such as in an E. coli expression system, as is well known in the art, and isolated in amounts useful for therapeutic compositions.

In embodiments, at least one therapeutic agent is disclosed that causes downregulation of palladin within targeted tissues. In embodiments, downregulation of palladin can lead to the disruption of cell structures and the death of cancer cells. In other embodiments, the at least one therapeutic agent is a small molecule that blocks the Palladin: Actin binding site.

In embodiments, the at least one therapeutic agent is a microRNA that targets the palladin gene. By targeting the palladin gene, the microRNA can reduce the production of palladin protein and disrupt cell structures. In embodiments, the at least one therapeutic agent is a combination of two or more of the above therapeutic agents. The combination of therapeutic agents can provide a more effective treatment for glioma.

As described above, a method of treating brain tumors is herein disclosed wherein a therapeutic agent causes downregulation of paladin. In embodiments, said agent comprises miR-96/miR-182, bound to gold nanoparticles and embedded in a hydrogel. Notably, in some embodiments SRC-mediated, platelet-derived growth factor-induced membrane ruffling and lamellipodia formation required both palladin and SPIN90. Taken together, it is reasonable to assume that downregulation of palladin or inhibition of its binding to the actin cytoskeleton might curb the aggressive phenotype of glioma tumors.

### EXAMPLES

### Example 1: Genomic, Bulk Gene Expression, Clinical Data, and Survival Analysis

### Materials and Methods

Genomic, bulk gene expression, clinical data, and overall survival of tumor and healthy samples were in whole or part based upon data generated from The Cancer Genome Atlas Pan-Cancer (TCGA-PANCAN), TCGA Glioblastoma Multiforme and Lower Grade Glioma (TCGA-GBMLGG), and the Genotype-Tissue Expression (GTEx) datasets. Analysis and visualization were performed using either BioRender or GraphPad Prism 9.3.1 (Graphpad Software, CA, USA). Multivariate Cox regression analysis was done via in-house scripts using R version 4.1.1.

### Cell Culture

Murine glioblastoma stem cells 005 and 007 were grown in media [e.g., DMEM/F12 media 103 (Gibco)], supplemented with GlutaMAX (1:20, 1:40, 1:60, 1:100, 1:120, or 1:40; preferably 1:200) (Gibco), 50-200 units/mL penicillin (preferably 100 units/mL), 10-100 mg/mL 104 streptomycin (preferably 50 mg/mL), N2 supplement 1:100 (Gibco), 2.5 μg/mL heparin (sigma), ng/ml FGF 105 (Peprotech), and 20 ng/mL EGF (Peprotech). and AFFR53 and AGR53 murine glioblastoma cells were grown in Dulbecco's Modified Eagle's Medium, high glucose (Biological Indus-107 tries) supplemented with 10% FBS (Biological Industries), 2 mM sodium pyruvate (Biolog-108 ical Industries), 100 units/mL penicillin and 50 mg/mL streptomycin. Cells were incubated 109 at 37° C. in a 5% CO2 atmosphere. Before use, each cell line was confirmed to have no mycoplasma contamination using the EZ-PCR mycoplasma test kit (Biological Industries).

RNA Extraction and Quantitative Reverse Transcription-Polymerase Chain Reaction (qRT-PCR)

Total RNA from cell lines was extracted using TRIzol reagent according to the manufacturer's instructions (Invitrogen, Thermo Fisher Scientific). Reverse transcription reaction was conducted using High-Capacity cDNA Reverse Transcription Kit with random primers (ABI). mRNA expression was tested using SYBR Green PCR Master Mix (ABI). PCR amplification and reading was done in triplicates using the StepOnePlus thermal cycler (ABI). Pallaind expression values were calculated based on the comparative threshold cycle (Ct) method and normalized to glyceraldehyde 3-phosphate dehydrogenase (GAPDH).

### Animal Studies and Fluorescent Confocal Microscopy of Mouse Glioblastoma Tumors

C57BL/6J female mice were purchased from Envigo Jerusalem Israel. All experiments involving animals were approved by the Tel Aviv University Institutional Animal Care and Use Committee. All mice, females and males used in this study were 8-16 weeks old when tumors were induced and bred under pathogen-free conditions. All animals were housed in individually ventilated cages (5 mice per cage) with autoclaved ASPEN wood chips bedding and provided with food and drinking water ad libitum with 10/10, 12/12, or 14/14-hour light/dark cycle (preferably 12/12-hour light/dark cycle). A total of 3×10⁵ 005 cells stably expressing enhanced green fluorescent protein (GFP) were stereotaxically injected into the hippocampus of the mice. After allowing the tumors to develop for 20-50, 27-35, or 30-33 days (preferably 27-35 days), mice were perfused with 1×PBS and fixed with 4% paraformaldehyde. Brains were collected and coronal sections (30-40 μm) were cut using a HM450 Microtome (ThermoFisher Scientific). Floating sections were blocked for 2 hours using a goat anti-mouse-HRP antibody (Jackson ImmnoResearch, Cat No. 115-035-166, Dilution 1:100) and then incubated overnight at 3-5° C. (preferably 4° C.) with a mouse anti-palladin monoclonal antibody (Novus, Cat No. NBP1-25959, Dilution: 1:100), followed by goat anti-mouse-AlexaFluor647 (Abcam, Cat No. ab150115, dilution 1:100). Nuclei were counterstained with DAPI (Molecular Probes) at 1 μg/ml. Images were acquired using a Leica TCS SP8 confocal microscope (Leica Microsystems, Wetzlar, Germany) and analyzed using Fiji/ImageJ 1.530.

### Immunohistochemical Staining of Palladin in Human Multi-Tissue Arrays

Formalin fixed paraffin embedded human tissue microarray (TMA) with CNS pathologies were obtained from US Biomax, Inc. (GL2081) and stained using BOND-III (Leica Biosystems). Anti-palladin primary antibody was diluted 1:100 and incubated for 1.5 hours. Post primary antibodies and polymer were both added and incubated for 1 hour one at a time. Last, 3,3′-diaminobenzidine (DAB) was added followed by hematoxylin counterstain. The slide was analyzed by a pathologist. All the incubation steps were carried out at room temperature using reagents in standard supplied with BOND-III.

### Single-Cell Gene Expression Data

Single-cell gene expression data of astrocytoma and glioblastoma multiforme (GBM) studies were accessed via the Single Cell Portal. For the purpose of finding genes similar to palladin, GeneCardsSuite's GeneLikeMe server was used. Analysis and visualization were done via in-house scripts using R version 4.1.1 packages and written in RStudio version 1.4.1717.

### In-Silico Flow Cytometry of Astrocytoma Tumors

To impute cell fractions in IDH1-mutated astrocytoma tumors, we used the CIBERSORTx server. Single cell RNA-seq of IDH-mutant astrocytoma data was downloaded from the Single Cell Portal and used to create a signature matrix. This signature matrix was then employed to impute cell fractions in samples from IDH1-mutated astrocytoma tumors from the TCGA-LGG dataset. Finally, differences in cell fractions were analyzed between tumors of different histological grades.

Enrichment Analysis of Gene Co-Expression with Palladin

Co-expression data were accessed and downloaded from the SEEK [Human] server. We conducted two distinct queries with PALLD as the query gene, one limiting the search space to astrocytoma-related datasets and the other limiting to GBM-related datasets. We then filtered out genes with a |co-expression Z score|>=1 and P<0.01. Analysis and visualization were done via in-house scripts using R version 4.1.1 packages, and written in RStudio version 1.4.1717.

### Statistical Analyses

Data are presented as violin plots or bar plots with median or mean±SEM (standard error of the mean), respectively, as was calculated by GraphPad Prism 9.3.1, which was also used for visualization. Statistical Analyses of gene expression and clinical data included initial outlier identification and removal using the ROUT (Q=1%) method followed by the Shapiro-Wilk normality test. Unpaired t and Mann-Whitney tests were used to compare between groups. Ordinary one-way ANOVA and Kruskal-Wallis tests were used in analyses of more than two groups, together with Holm-Šídak and Dunn's multiple comparison test, respectively. In all the tests, an α=0.05 was used.

1. A method of treating brain tumors in a subject in need thereof, the method comprising:
a) detecting or diagnosing glioma in the subject, wherein detecting or diagnosing glioma comprises:
measuring PALLD mRNA levels in a plasma sample of the subject;
correlating the PALLD mRNA levels to PALLD mRNA plasma levels corresponding to a likelihood of having a specific type of glioma;
making a diagnosis based on the PALLD mRNA levels of the plasma sample as compared to the expected PALLD mRNA plasma levels corresponding to a likelihood of having a specific type of glioma; and
b) treating subject diagnosed with specific type of glioma or having the likelihood of developing specific type of glioma with at least one therapeutic agent for treating glioma.

2. The method of treating brain tumors of claim 1, wherein:
the at least one therapeutic agent causes downregulation of palladin within targeted tissue of a subject.

3. The method of treating brain tumors of claim 2, wherein:
the at least one therapeutic agent causing downregulation of palladin comprises at least one microRNA.

4. The method of treating brain tumors of claim 3, wherein:
the at least one therapeutic agent causing downregulation of palladin comprises at least one microRNA, wherein the at least one microRNA is bound to gold nanoparticles.

5. The method of treating brain tumors of claim 4, wherein:
the at least one therapeutic agent causing downregulation of palladin comprises the least one microRNA, wherein the least one microRNA are bound to gold nanoparticles and are further embedded in a hydrogel.

6. The method of claim 1, wherein the therapeutic agent comprises at least one miRNA, or at least one vector expressing or encoding the same, for use in reducing or preventing the specific type of glioma.

7. The method of claim 6, wherein the therapeutic agent is formulated for systemic administration.

8. The method of claim 6, wherein the therapeutic agent is formulated for local administration.

9. The method of claim 8, wherein the therapeutic agent is formulated for intra-tumor administration.

10. The method of claim 6, wherein the glioma comprises a cancer associated with an abnormal palladin expression and/or activity.

11. The method of claim 6, wherein the glioma is selected from the group consisting of astrocytoma or oligodendroglioma.

12. The method of claim 6, wherein the glioma comprises a glioblastoma.

13. The method of claim 6, wherein the subject is at risk of developing metastasis and the administering is carried out prior to metastasis formation.

14. The method of claim 6, wherein the subject has already developed metastases and the administering is carried out after metastasis formation.

15. The method of claim 6, wherein the glioma comprises an ependymoma.

16. The method of treating brain tumors in claim 1, wherein:
the therapeutic agent inhibits the binding of palladin to actin cytoskeleton.

17. The method of treating brain tumors in claim 1, wherein:
glioblastoma tumors express approximately 150% and 70% more palladin than oligodendroglioma, and astrocytoma tumors, respectively (P<0.0001 and P<0.0001).

18. A method of detecting or diagnosing glioma tumor type in a subject, the method comprising:
measuring PALLD mRNA levels in a plasma sample of the subject;
correlating the PALLD mRNA levels to PALLD mRNA plasma levels corresponding to a likelihood of having a specific type of glioma;
making a diagnosis based on the PALLD mRNA levels of the plasma sample as compared to the expected PALLD mRNA plasma levels corresponding to a likelihood of having a specific type of glioma.

19. The method of detecting or diagnosing glioma tumor type of claim 18, wherein the specific type of glioma comprises an adult-type diffuse glioma.

20. A method of delineating brain tumor tissue from surrounding brain, the method comprising:
injecting a palladin-associating fluorophore or prodrug comprising a palladin-associating fluorophore into a subject for fluorescence-guided neurosurgery.
